SEQUENCE LISTING (1 sequence)

SEQ ID NO: 1 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

(19) United States
(12) Patent Application Publication
(10) Pub. No.: US 2024/0238460 A1
(43) Pub. Date: Jul. 18, 2024

(54) IMPROVED PROSTATE-SPECIFIC MEMBRANE ANTIGEN TARGETING RADIOPHARMACEUTICALS AND USES THEREOF

(71) Applicant: TELIX PHARMACEUTICALS (INNOVATIONS) PTY LTD, Melbourne VIC (AU)

(72) Inventors: Frederik GIESEL, Heidelberg (DE); Jens CARDINALE, Ludwigshafen (DE); Clemens KRATOCHWIL, Weinheim (DE); Uwe HABERKORN, Schwetzingen (DE)

(21) Appl. No.: 18/563,673

(22) PCT Filed: May 31, 2022
(86) PCT No.: PCT/EP2022/064668

§ 371 Date: Nov. 22, 2023

(30) Foreign Application Priority Data
May 31, 2021 (EP) 21176899.9

(51) Int. Cl. A61K 51/08; A61P 35/00

(52) U.S. Cl. CPC A61K 51/088; A61P 35/00

(57) ABSTRACT

The present invention relates to diagnosis and treatment of malignancies characterised by prostate-specific membrane antigen (PSMA) expression. The invention particularly relates to improved radiopharmaceuticals which selectively bind to PSMA and are suitable for planar imaging of PSMA expression in subjects to diagnose and/or monitor malignancies wherein PSMA is (over)expressed. Additionally, the invention relates to improved radiopharmaceuticals which selectively bind to PSMA and are suitable to act as radionuclide treatment agents. The radiopharmaceuticals rely on a pharmacophore capable of interacting with PSMA and N-terminal mercaptoacetyltripeptides capable of coordinating radioactive metals such as technetium and rhenium.

### FIELD OF THE INVENTION

The present invention relates to the field of radiopharmaceuticals. In particular. the radiopharmaceuticals are capable of selective binding to prostate-specific membrane antigens (PSMA) and can be used in both diagnosis and treatment of cancer types that are accompanied by (over)expression of PSMA. The PSMA radiopharmaceuticals described herein are characterised by a number of advantages over PSMA radiopharmaceuticals known in the art.

### BACKGROUND OF THE INVENTION

In 2018, over 1 million of new prostate cancer cases were registered worldwide. rendering the disease the second most frequent malignancy in adult men worldwide (Rawla. World J Oncol. 2019). Both the incidence and mortality rate of prostate cancer correlate with increasing age, and the average age of diagnosis is about 66 years. Albeit significant regional differences can be discerned. the global mortality rate of prostate cancer relates to about 3.8% of all deaths caused by cancer in men (Jemal et al., CA Cancer J Clin. 2018). Given the often asymptomatic first stages of prostate cancer progression. the regional incidence rates are tightly correlated to both the adoption of screening campaigns, an increase in average life expectancy. but also westernisation of the lifestyle which impacts obesity, physical inactivity, and dietary factors (Baade et al., Mol Nutr Food Res. 2009). Hence, it is expected that incidence rates will continue to rise up to 2040 (Ferlay et al., Cancer Today, IARC Cancerbase. 2018).

Continuous advancements in the fields of medicine and health sciences are unearthing new diagnostic markers and therapeutic targets to combat diseases. A marker of particular interest in the context of prostate cancer is the enzyme glutamate carboxypeptidase II, commonly referred to in the art as prostate-specific membrane antigen (PSMA). PSMA is consistently expressed in all types of prostate tissue and highly overexpressed in prostate cancer tissue and it has been demonstrated that PSMA expression levels are directly correlated to androgen independence, metastasis, and disease progression (Santoni et al., J Biol Regul Homeost Agents. 2014). In view hereof, diagnosis and monitoring of prostate cancer by positron emission tomography (PET) or by single-photon emission tomography (SPECT) of PSMA is increasingly used in clinical settings (Fanti et al., Eur J Nucl Med Mol Imaging, 2021). Alternatively, the highly selective expression profile of PSMA translates to PSMA being especially suited as target for a radionuclide therapy of prostate cancer and other malignancies that are accompanied by PSMA (over)expression. In addition to the highly specific (high) expression levels that can be measured for PSMA in (malignant) prostate tissue. PSMA is rapidly internalised by cells upon ligand binding, which leads to a further concentration of the radionuclide molecule inside cells, thus increasing tumour absorbed dose. Consequently, a number of radiopharmaceuticals have been developed that comprise a selective PSMA ligand conjugated to a radiometal which aim to achieve optimal PSMA imaging and/or PSMA radionuclide therapy. So far, in the field of diagnosis the PSMA targeting PET tracers gained most attraction, while in the field of therapy recent research focussed on Lu-177 and Ac-225 based radiopharmaceuticals. However, worldwide nuclear medical infrastructure is far more developed in the field of SPECT. Thus, a theragnostic tandem applying Tc-99m and Re-188 labeled PSMA tracers would be highly desirable - especially since both radionuclides are available from common generator systems which are authorized for medical use in most countries. For pure diagnostic application (SPECT) a number of ^{99m}Tc-radiotracers like iPSMA have been developed. However, particularly in the case of iPSMA, the HYNIC chelator renders ¹⁸⁸Re-labelling at least difficult and is, thus, not a viable basis for a potential “kit application”. Further, improving the body clearance of the radiopharmaceutical would be equally desirable.

In view hereof, any approach that may facilitate diagnosis and/or improve treatment of prostate cancer is a valuable asset for identifying afflicted men and/or lowering the mortality rate of men diagnosed with prostate cancer.

### SUMMARY OF THE INVENTION

Through extensive research and experimentation, the inventors have identified novel PSMA radiopharmaceuticals or radiotheranostics (radiotherapeutics or radiodiagnostics) with improved properties vis-à-vis known PSMA radiopharmaceuticals. The radiopharmaceuticals described herein, including but not limited to Technetium-99m (^{99m}Tc) labeled imaging agents and Rhenium-188 (¹⁸⁸Re) labeled therapeutics/thera(g)nostics, are characterized by structural modifications in the linker region and/or the chelator and have improved pharmacokinetic properties while maintaining a stable ¹⁸⁸ Re/^{99m}Tc-coordination to the molecule. The pharmacophore presents three carboxylic groups able to interact with the respective side chains of PSMA and an oxygen as part of zinc complexation in the active center. Besides these obligatory interactions, the inventors were able to optimize the lipophilic interactions in the linker region compared to the lead structure ^{99m}Tc-EDDA-HYNIC-iPSMA (TLX-598/[^{99m}Tc]Tc-TLX-598—cf. WO 2017/222362). Moreover, the inventors replaced the HYNIC chelator by N-terminal mercaptoacetyltripeptides, which are (also) capable of coordinating Technetium-99m and Rhenium-188 (in form of the so called “oxo-core” Tc(V)O/Re(V)O, coordinated towards the three amide bonds and the deprotonated sulfur; reference). In contrast, this type of coordination does not need application of a stabilizing co-ligand (e.g., EDDA) and, thus, represents a simplification of the underlying chemistry. Additionally, the modified radiopharmaceuticals described herein display a good cellular uptake and renal clearance rates. The exemplified ^{99m}Tc and ¹⁸⁸Re radiopharmaceuticals hence present improved radiopharmaceutical molecules for respectively planar imaging and radionuclide therapy of PSMA positive tumors.

The invention therefore provides the following aspects:

Aspect 1. A first aspect of the present invention provides a labeling precursor in the form of a compound of formula (I) or a stereoisomer, or tautomer thereof,

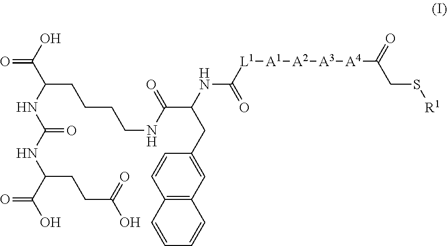

wherein,
R¹ is selected from the group consisting of hydrogen, —C(O)R¹¹, —C(O)₂R¹², —C(O)NR¹²R¹³, —SR¹⁰, C₁₋₆alkyl, arylC₁₋₆alkyl, heteroarylC₁₋₆alkyl, heterocyclylC₁₋₆alkyl, aryl, heteroaryl, heterocyclyl; wherein said C₁₋₆alkyl, arylC₁₋₆alkyl, heteroarylC₁₋₆alkyl, heterocyclylC₁₋₆alkyl, aryl, heteroaryl, or heterocyclyl can be unsubstituted or substituted with one or more Z¹;
L¹ is

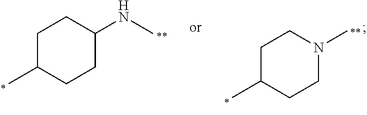

wherein * represents where L¹ is bound to the carbonyl group; and ** represents where L¹ is bound to A¹;
A¹ is

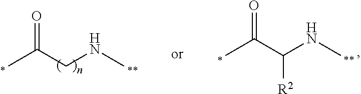

wherein * represents where A¹ is bound L¹; and ** represents where A¹ is bound to A²; and wherein,
n is an integer selected from 1, 2, 3, 4 or 5;
R² is selected from the group consisting of hydroxyC₁₋₆alkyl, C₁₋₆alkylNHR³ C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkyIS(O)CH₃, C₁₋₆alkylS(CH₃)₂⁺, C₁₋₆alkylNHC(O)heterocycle and C₁₋₆alkylC(O)NH₂;
R³ is H or —C(O)(CH₂)SH;
A² is selected from:

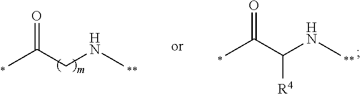

wherein * represents where A² is bound A¹; and ** represents where A² is bound to A³; or wherein A² is absent; and wherein,
m is an integer selected from 1, 2, 3, 4 or 5;
R⁴ is selected from the group consisting of hydroxyC₁₋₆alkyl, C₁₋₆alkylNHR⁵, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂;
R⁵ is H or —C(O)(CH₂)SH;
A³ is selected from:

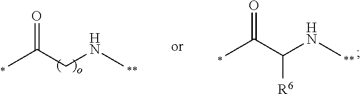

wherein * represents where A³ is bound A²; and ** represents where A³ is bound to A⁴; or wherein A³ is absent; and wherein,
o is an integer selected from 1, 2, 3, 4 or 5;
R⁶ is selected from the group consisting of hydroxyC₁₋₆alkyl, C₁₋₆alkylNHR⁷, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂;
R⁷ is H or —C(O)(CH₂)SH;
A⁴ is selected from:

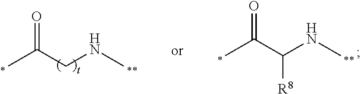

wherein * represents where A⁴ is bound A³; and ** represents where A⁴ is bound to the carbonyl group; or wherein A⁴ is absent; and wherein,
t is an integer selected from 1, 2, 3, 4 or 5;
R⁸ is selected from the group consisting of hydroxyC₁₋₆alkyl, C₁₋₆alkylNHR⁹, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂;
R⁹ is H or —C(O)(CH₂)SH;
R¹⁰ is selected from the group consisting of C₁₋₆alkyl, heterocycle, aryl, and heteroaryl;
or R¹⁰ is a group of formula (i);

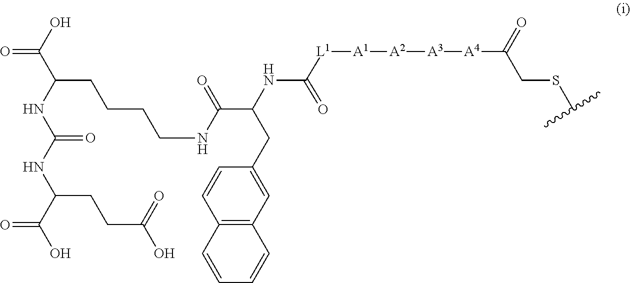

wherein the wavy line ( [symbol] ) indicates the point of attachment to the S atom and L¹, A¹, A², A³, and A⁴ are as defined for structure (I);
each R¹¹ is independently selected from the group consisting of C₁₋₆alkyl, haloC₁₋₆alkyl, aryl, haloC₁₋₆alkyl, arylC₁₋₆alkyl, heterocyclyl, heteroaryl;
each R¹² is independently selected from the group consisting of hydrogen, C₁₋₆alkyl, aryl, haloC₁₋₆alkyl, CH₂CCl₃, CH₂OCH₃, arylC₁₋₆alkyl, heterocyclyl, heteroaryl;
each R¹³ is independently selected from the group consisting of hydrogen, C₁₋₆alkyl, aryl, haloC₁₋₆alkyl, arylC₁₋₆alkyl, heterocyclyl, heteroaryl;
each Z¹ is independently selected from the group consisting of —OR¹¹, —C(O)R¹¹, nitro, hydroxyl, C₁₋₆alkyl, aryl, heteroaryl, —SR¹¹, —NR¹²C(O)R¹³, —C(O)₂R¹², cyano, —S(O)₂R¹⁰, halo, haloC₁₋₆alkyl, haloC₁₋₆alkyloxy, heterocyclyl, amino, —NR¹¹R¹², —C(O)NR¹²R¹³, —S(O)R¹⁰, —S(O)₂NR¹²R¹³; wherein said C₁₋₆alkyl, aryl, can be unsubstituted or substituted with one or more C₁₋₄alkyl, methoxy, nitro, —C(O)aryl, halo, trifluoromethyl, trifluoromethoxy.

Aspect 2. In a second aspect the present invention provides a labeling precursor in the form of a compound of formula (I) or a stereoisomer, or tautomer thereof,
wherein,
R¹ is selected from the group consisting of hydrogen, —C(O)R¹¹, —C(O)₂R¹², —C(O)NR¹²R¹³, —SR¹⁰, C₁₋₆alkyl, arylC₁₋₆alkyl, heteroarylC₁₋₆alkyl, heterocyclylC₁₋₆alkyl, aryl, heteroaryl, heterocyclyl; preferably R¹ is hydrogen, —C(O)R¹¹, —C(O)₂R¹², —C(O)NR¹²R¹³, —SR¹⁰, C₁₋₆alkyl, arylC₁₋₆alkyl, heteroarylC₁₋₆alkyl; preferably R¹ is hydrogen, —C(O)R¹¹, —C(O)₂R¹², —C(O)NR¹²R¹³, —SR¹⁰, C₁₋₆alkyl, arylC₁₋₆alkyl;
wherein said C₁₋₆alkyl, arylC₁₋₆alkyl, heteroarylC₁₋₆alkyl, heterocyclylC₁₋₆alkyl, aryl, heteroaryl, or heterocyclyl can be unsubstituted or substituted with one or more Z¹; preferably said groups are unsubstituted or substituted with one, two or three Z¹;
L¹ is

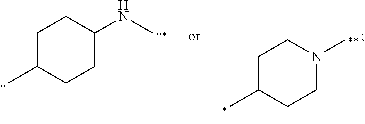

wherein * represents where L¹ is bound to the carbonyl group; and ** represents where L¹ is bound to A¹;
A¹ is

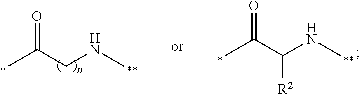

wherein * represents where A¹ is bound L¹; and ** represents where A¹ is bound to A²; and wherein,
n is an integer selected from 1, 2, 3, 4 or 5; preferably n is selected from 1, 2, or 3;
R² is selected from the group consisting of hydroxyC₁₋₆alkyl, C₁₋₆alkylNHR³, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂; preferably R² is hydroxyC₁₋₆alkyl, C₁₋₆alkylNHR³, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylSeH, C₁₋₆alkylS(O)CH₃, C₁₋₆alkylS(CH₃)₂⁺, and C₁₋₆alkylC(O)NH₂; preferably R² is hydroxyC₁₋₆alkyl, C₁₋₆alkylNHR₃, C₁₋₆alkylC(O)OH, C₁₋₆alkylNHC(NH)NH₂, hydrogen, C₁₋₆alkyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylS(O)CH₃, C₁₋₆alkylS(CH₃)₂⁺, and C₁₋₆alkylC(O)NH₂; preferably R² is —CH₂OH, —CH₂NHR₃, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R³ is H or —C(O)(CH₂)SH;
A² is selected from:

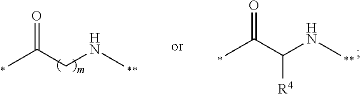

wherein * represents where A² is bound A¹; and ** represents where A² is bound to A³; or wherein A² is absent; and wherein,
m is an integer selected from 1, 2, 3, 4 or 5;
R⁴ is selected from the group consisting of hydroxyC₁₋₆alkyl, C₁₋₆alkylNHR⁵, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂; preferably R⁴ is hydroxyC₁₋₆alkyl, C₁₋₆alkylNHR⁵, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylSeH, C₁₋₆alkylS(O)CH₃, C₁₋₆alkylS(CH₃)₂⁺, and C₁₋₆alkylC(O)NH₂; preferably R⁴ is hydroxyC₁₋₆alkyl, C₁₋₆alkylNHR⁵, C₁₋₆alkylC(O)OH, C₁₋₆alkylNHC(NH)NH₂, hydrogen, C₁₋₆alkyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylS(O)CH₃, C₁₋₆alkylS(CH₃)₂⁺, and C₁₋₆alkylC(O)NH₂; preferably R⁴ is —CH₂OH, —CH₂NHR₃, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R⁵ is H or —C(O)(CH₂)SH;
A³ is selected from:

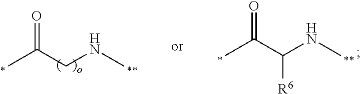

wherein * represents where A³ is bound A²; and ** represents where A³ is bound to A⁴; or wherein A³ is absent; and wherein,
o is an integer selected from 1, 2, 3, 4 or 5;
R⁶ is selected from the group consisting of hydroxyC₁₋₆alkyl, C₁₋₆alkylNHR⁷, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂; preferably R⁶ is hydroxyC₁₋₆alkyl, C₁₋₆alkylNHR⁷, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylSeH, C₁₋₆alkylS(O)CH₃, C₁₋₆alkylS(CH₃)₂⁺, and C₁₋₆alkylC(O)NH₂; preferably R⁶ is hydroxyC₁₋₆alkyl, C₁₋₆alkylNHR⁷, C₁₋₆alkylC(O)OH, C₁₋₆alkylNHC(NH)NH₂, hydrogen, C₁₋₆alkyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylS(O)CH₃, C₁₋₆alkylS(CH₃)₂⁺, and C₁₋₆alkylC(O)NH₂; preferably R⁶ is —CH₂OH, —CH₂NHR³, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R⁷ is H or —C(O)(CH₂)SH;
A⁴ is selected from:

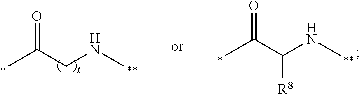

wherein * represents where A⁴ is bound A³; and ** represents where A⁴ is bound to the carbonyl group; or wherein A⁴ is absent; and wherein,
t is an integer selected from 1, 2, 3, 4 or 5;
R⁸ is selected from the group consisting of hydroxyC₁₋₆alkyl, C₁₋₆alkylNHR⁹, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂; preferably R⁸ is hydroxyC₁₋₆alkyl, C₁₋₆alkylNHR⁹, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylSeH, C₁₋₆alkylS(O)CH₃, C₁₋₆alkylS(CH₃)₂⁺, and C₁₋₆alkylC(O)NH₂; preferably R⁸ is hydroxyC₁₋₆alkyl, C₁₋₆alkylNHR⁹, C₁₋₆alkylC(O)OH, C₁₋₆alkylNHC(NH)NH₂, hydrogen, C₁₋₆alkyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylS(O)CH₃, C₁₋₆alkylS(CH₃)₂⁺, and C₁₋₆alkylC(O)NH₂; preferably R⁸ is —CH₂OH, —CH₂NHR₃, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R⁹ is H or —C(O)(CH₂)SH;
R¹⁰ is selected from the group consisting of C₁₋₆alkyl, heterocycle, aryl, and heteroaryl;
or R¹⁰ is a group of formula (i);

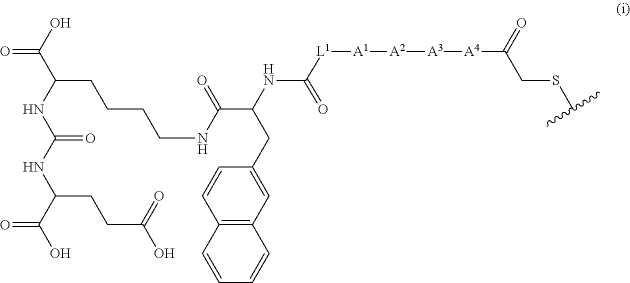

wherein the wavy line ( [symbol] ) indicates the point of attachment to the S atom and L¹, A¹, A², A³, and A⁴ are as defined for structure (I); preferably R¹⁰ is C₁₋₆alkyl, or a group of formula (i);
each R¹¹ is independently selected from the group consisting of C₁₋₆alkyl, haloC₁₋₆alkyl, aryl, arylC₁₋₆alkyl, heterocyclyl, heteroaryl; preferably each R¹¹ is C₁₋₆alkyl, haloC₁₋₆alkyl, aryl, and arylC₁₋₆alkyl; each R¹² is independently selected from the group consisting of hydrogen, C₁₋₆alkyl, aryl, haloC₁₋₆alkyl, CH₂CCl₃, CH₂OCH₃, arylC₁₋₆alkyl, heterocyclyl, heteroaryl; preferably each R¹² is hydrogen, C₁₋₆alkyl, aryl, CH₂CCl₃, CH₂OCH₃;
each R¹³ is independently selected from the group consisting of hydrogen, C₁₋₆alkyl, aryl, haloC₁₋₆alkyl, arylC₁₋₆alkyl, heterocyclyl, heteroaryl; preferably each R¹² is hydrogen, C₁₋₆alkyl, haloC₁₋₆alkyl, aryl, and arylC₁₋₆alkyl; each Z¹ is independently selected from the group consisting of —OR¹¹, —C(O)R¹¹, nitro, hydroxyl, C₁₋₆alkyl, aryl, heteroaryl, —SR¹¹, —NR¹²C(O)R¹³, —C(O)₂R¹², cyano, —S(O)₂R¹⁰, halo, haloC₁₋₆alkyl, haloC₁₋₆alkyloxy, heterocyclyl, amino, —NR¹¹R¹², —C(O)NR¹²R¹³, —S(O)R¹⁰, —S(O)₂NR¹²R¹³; preferably each Z¹ is —OR¹¹, —C(O)R¹¹, nitro, hydroxyl, C₁₋₆alkyl, aryl, heteroaryl, —SR¹¹, —NR¹²C(O)R¹³, —C(O)₂R¹², cyano, —S(O)₂R¹⁰, haloC₁₋₆alkyl, amino, —C(O)NR¹²R¹³, —S(O)R¹⁰, —S(O)₂NR¹²R¹³; preferably each Z¹ is —OR¹¹, —C(O)R¹¹, nitro, hydroxyl, C₁₋₆alkyl, aryl, heteroaryl, —SR¹¹, —NR¹²C(O)R¹³, —C(O)₂R¹², cyano, —S(O)₂R¹⁰, haloC₁₋₆alkyl;
wherein said C₁₋₆alkyl, or aryl, can be unsubstituted or substituted with one or more C₁₋₄alkyl, methoxy, nitro, —C(O)aryl, halo, trifluoromethyl, trifluoromethoxy; preferably said groups can be unsubstituted or substituted with one, two or three C₁₋₄alkyl, methoxy, nitro, —C(O)aryl, halo, trifluoromethyl, trifluoromethoxy; preferably said groups can be unsubstituted or substituted with one or more C₁₋₄alkyl, methoxy, nitro, —C(O)aryl, halo;

Aspect 3. In a third aspect the present invention provides a labeling precursor in the form of a compound of any one of formula (IA), (IB), (IC) or (ID):

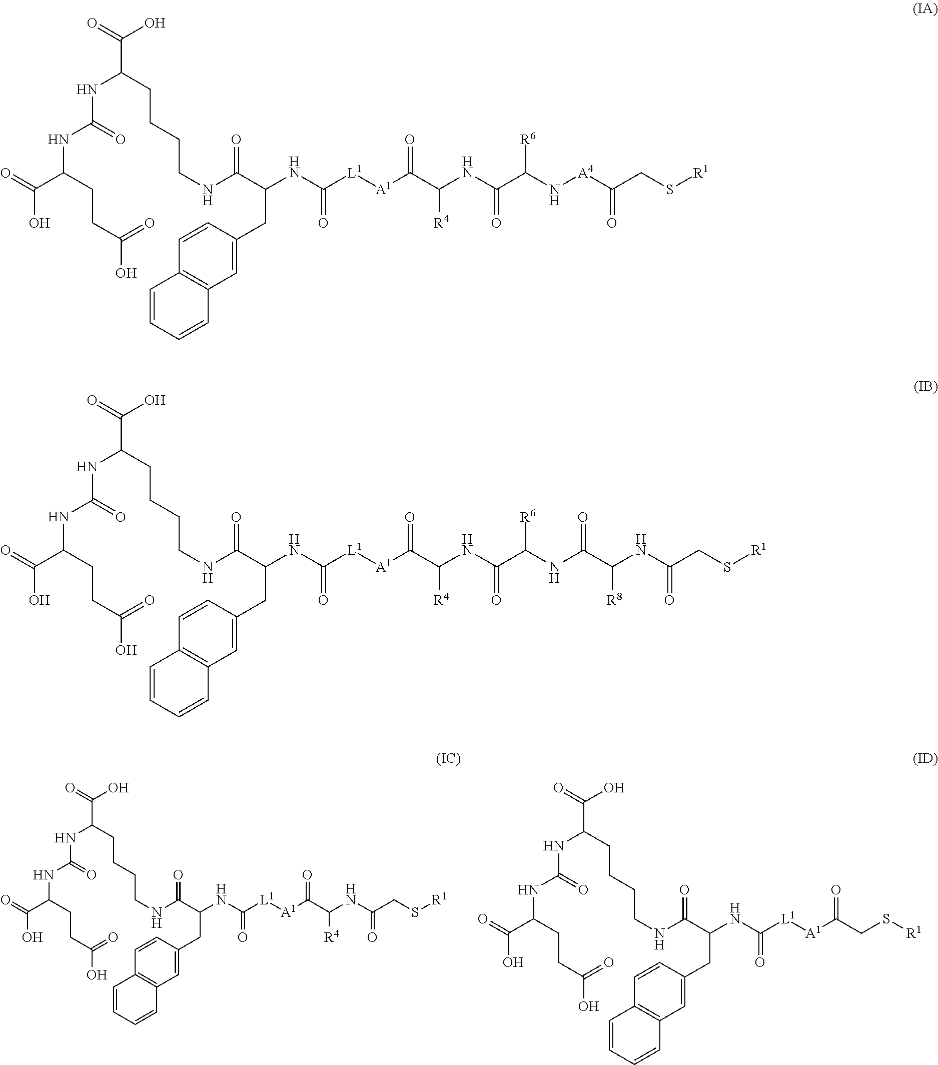

wherein L¹, A¹, A⁴, R¹, R⁴, R⁶ and R⁸ have the same meaning as that defined herein.

Aspect 4. According to particular embodiments, the present invention provides a compound of formula (I),
wherein,
R² is selected from the group consisting of —CH₂OH, —CH₂NHR³, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R⁴ is selected from the group consisting of —CH₂OH, —CH₂NHR⁵, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R⁶ is selected from the group consisting of —CH₂OH, —CH₂NHR⁷, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R⁸ is selected from the group consisting of —CH₂OH, —CH₂NHR⁹, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
preferably wherein, R¹ is hydrogen, acetyl or —SR¹⁰, wherein R¹⁰ is a group of formula (i).

Aspect 5. According to particular embodiments, the present invention provides a labeling precursor in the form of a compound of formula (I),
wherein,
R¹ is selected from the group consisting of hydrogen, —C(O)R¹¹, —C(O)₂R¹², —C(O)NR¹²R¹³, —SR¹⁰, C₁₋₆alkyl, arylC₁₋₆alkyl, heteroarylC₁₋₆alkyl, heterocyclylC₁₋₆alkyl, aryl, heteroaryl, heterocyclyl; preferably R¹ is hydrogen, C₁₋₆alkylcarbonyl, arylC₁₋₄alkylcarbonyl, arylcarbonyl, C₁₋₆alkyloxycarbonyl, arylC₁₋₆alkyloxycarbonyl, aryloxycarbonyl, heteroaryloxy, aminocarbonyl, mono-C₁₋₆alkylaminocarbonyl, di-C₁₋₆alkylaminocarbonyl, mono-arylaminocarbonyl, di-alrylaminocarbonyl, C₁₋₆alkylthio, arylC₁₋₆alkylthio, arylthio, C₁₋₆alkyl, arylC₁₋₆alkyl, heteroarylC₁₋₆alkyl, heterocyclylC₁₋₆alkyl, aryl, heteroaryl, heterocyclyl; preferably R¹ is hydrogen, C₁₋₆alkylcarbonyl, arylC₁₋₆alkylcarbonyl, arylcarbonyl, C₁₋₆alkyloxycarbonyl, arylC₁₋₆alkyloxycarbonyl, aryloxycarbonyl, mono-C₁₋₆alkylaminocarbonyl, di-C₁₋₆alkylaminocarbonyl, C₁₋₆alkylthio, arylC₁₋₆alkylthio, arylthio, C₁₋₆alkyl, arylC₁₋₆alkyl, heteroarylC₁₋₆alkyl, heterocyclylC₁₋₆alkyl, aryl, heteroaryl, heterocyclyl; preferably R¹ is hydrogen, C₁₋₆alkylcarbonyl, arylC₁₋₆alkylcarbonyl, arylcarbonyl, C₁₋₆alkyloxycarbonyl, arylC₁₋₆alkyloxycarbonyl, aryloxycarbonyl, C₁₋₆alkylthio, arylC₁₋₆alkylthio, arylthio, C₁₋₆alkyl, arylC₁₋₆alkyl; preferably R¹ is hydrogen, C₁₋₄alkylcarbonyl, arylC₁₋₄alkylcarbonyl, arylcarbonyl, C₁₋₄alkyloxycarbonyl, arylC₁₋₄alkyloxycarbonyl, aryloxycarbonyl, C₁₋₄lkylthio, arylC₁₋₄alkylthio, arylthio, C₁₋₄alkyl, arylC₁₋₄alkyl;
wherein said groups can be unsubstituted or substituted with one or more Z¹; preferably said groups are unsubstituted or substituted with one, two or three Z¹.

Aspect 6. According to particular embodiments, the present invention provides a labeling precursor in the form of a compound of formula (I),
wherein,
R² is selected from the group consisting of hydroxy C₁₋₆alkyl, C₁₋₆alkylNHR³, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂; preferably R² is hydroxyC₁₋₆alkyl, C₁₋₆alkylNHR³, C₁₋₆alkylC(O)OH, C₁₋₆alkylNHC(NH)NH₂, hydrogen, C₁₋₆alkyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylSeH, C₁₋₆alkylS(O)CH₃, C₁₋₆alkylS(CH₃)₂⁺, and C₁₋₆alkylC(O)NH₂; preferably R² is hydroxyC₁₋₄alkyl, C₁₋₄alkylNHR³, C₁₋₆alkylC(O)OH, C₁₋₄alkylNHC(NH)NH₂, hydrogen, C₁₋₄alkyl, aminoC₁₋₆alkyl, mercaptoC₁₋₆alkyl, C₁₋₄alkylthioC₁₋₄alkylene, —CH(OH)CH₃, —C(O)OH, C₁₋₄alkyl(CO₂H)₂, —SO₃H, C₁₋₄alkylSeH, C₁₋₄alkylS(O)CH₃, C₁₋ₑ₄salkylS(CH₃)₂⁺, and C₁₋₄alkylC(O)NH₂; preferably R² is hydroxyC₁₋₄alkyl, C₁₋₄alkylNHR³, C₁₋₆alkylC(O)OH, C₁₋₄alkylNHC(NH)NH₂, hydrogen, C₁₋₄alkyl, aminoC₁₋₆alkyl, —CH(OH)CH₃, —C(O)OH, C₁₋₄alkyl(CO₂H)₂, —SO₃H, C₁₋₄alkylS(O)CH₃, C₁₋ₑ₄alkylS(CH₃)₂⁺, and C₁₋₄alkylC(O)NH₂; preferably R² is —CH₂OH, —CH₂NHR³, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂.

Aspect 7. According to particular embodiments, the present invention provides a labeling precursor in the form of a compound of formula (I),
wherein,
R⁴ is selected from the group consisting of hydroxyC₁₋₆alkyl, C₁₋₆alkylNHR⁵, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂; preferably R⁴ is hydroxyC₁₋₆alkyl, C₁₋₆alkyINHR⁵, C₁₋₆alkylC(O)OH, C₁₋₆alkylNHC(NH)NH₂, hydrogen, C₁₋₆alkyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylSeH, C₁₋₆alkylS(O)CH₃, C₁₋₆alkylS(CH₃)₂⁺, and C₁₋₆alkylC(O)NH₂; preferably R⁴ is hydroxyC₁₋₄alkyl, C₁₋₄alkylNHR⁵, C₁₋₆alkylC(O)OH, C₁₋₄alkylNHC(NH)NH₂, hydrogen, C₁₋₄alkyl, aminoC₁₋₆alkyl, mercaptoC₁₋₆alkyl, C₁₋₄alkylthioC₁₋₄alkylene, —CH(OH)CH₃, —C(O)OH, C₁₋₄alkyl(CO₂H)₂, —SO₃H, C₁₋₄alkylSeH, C₁₋₄alkylS(O)CH₃, C₁₋ₑ₄alkylS(CH₃)₂⁺, and C₁₋₄alkylC(O)NH₂; preferably R⁴ is hydroxyC₁₋₄alkyl, C₁₋₄alkylNHR⁵, C₁₋₆alkylC(O)OH, C₁₋₄alkylNHC(NH)NH₂, hydrogen, C₁₋₄alkyl, aminoC₁₋₆alkyl, —CH(OH)CH₃, —C(O)OH, C₁₋₄alkyl(CO₂H)₂, —SO₃H, C₁₋₄alkylS(O)CH₃, C₁₋ₑ₄salkylS(CH₃)₂⁺, and C₁₋₄alkylC(O)NH₂; preferably R⁴ is —CH₂OH, —CH₂NHR³, —CH₂C(O)OH, —(CH₂)₃NHC(NH)NH₂, hydrogen, C₁₋₆alkyl, —(CH₂)₂OH. —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂.

Aspect 8. According to particular embodiments, the present invention provides a labeling precursor in the form of a compound of formula (I),
wherein,
R⁶ is selected from the group consisting of hydroxyC₁₋₆alkyl, C₁₋₆alkylNHR⁷, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂; preferably R⁶ is hydroxyC₁₋₆alkyl, C₁₋₆alkylNHR⁷, C₁₋₆alkylC(O)OH, C₁₋₆alkylNHC(NH)NH₂, hydrogen, C₁₋₆alkyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylSeH, C₁₋₆alkylS(O)CH₃, C₁₋₆alkylS(CH₃)₂⁺, and C₁₋₆alkylC(O)NH₂; preferably R⁶ is hydroxyC₁₋₄alkyl, C₁₋₄alkylNHR⁷, C₁₋₆alkylC(O)OH, C₁₋₄alkylNHC(NH)NH₂, hydrogen, C₁₋₄alkyl, aminoC₁₋₆alkyl, mercaptoC₁₋₆alkyl, C₁₋₄alkylthioC₁₋₄alkylene, —CH(OH)CH₃, —C(O)OH, C₁₋₄alkyl(CO₂H)₂, —SO₃H, C₁₋₄alkylSeH, C₁₋₄alkylS(O)CH₃, C₁₋ₑ₄alkylS(CH₃)₂⁺, and C₁₋₄alkylC(O)NH₂; preferably R⁶ is hydroxyC₁₋₄alkyl, C₁₋₄alkylNHR⁷, C₁₋₆alkylC(O)OH, C₁₋₄alkylNHC(NH)NH₂, hydrogen, C₁₋₄alkyl, aminoC₁₋₆alkyl, —CH(OH)CH₃, —C(O)OH, C₁₋₄alkyl(CO₂H)₂, —SO₃H, C₁₋₄alkylS(O)CH₃, C₁₋ₑ₄alkylS(CH₃)₂⁺, and C₁₋₄alkylC(O)NH₂; preferably R⁴ is —CH₂OH, —CH₂NHR³, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂.

Aspect 9. According to particular embodiments, the present invention provides a labeling precursor in the form of a compound of formula (I),
wherein,
R⁸ is selected from the group consisting of hydroxyC₁₋₆alkyl, C₁₋₆alkylNHR⁹, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂; preferably R⁸ is hydroxyC₁₋₆alkyl, C₁₋₆alkylNHR⁹, C₁₋₆alkylC(O)OH, C₁₋₆alkylNHC(NH)NH₂, hydrogen, C₁₋₆alkyl, aminoC₁₋₆alkyl, mercaptoC₁₋₆alkyl, C₁₋₆alkylthioC₁₋₆alkylene, —CH(OH)CH₃, —C(O)OH, C₁₋₆alkyl(CO₂H)₂, —SO₃H, C₁₋₆alkylSeH, C₁₋₆alkylS(O)CH₃, C₁₋₆alkylS(CH₃)₂, and C₁₋₆alkylC(O)NH₂; preferably R⁸ is hydroxyC₁₋₄alkyl, C₁₋₄alkylNHR⁹, C₁₋₆alkylC(O)OH, C₁₋₄alkylNHC(NH)NH₂, hydrogen, C₁₋₄alkyl, aminoC₁₋₆alkyl, mercaptoC₁₋₆alkyl, C₁₋₄alkylthioC₁₋₄alkylene, —CH(OH)CH₃, —C(O)OH, C₁₋₄alkyl(CO₂H)₂, —SO₃H, C₁₋₄alkylSeH, C₁₋₄alkylS(O)CH₃, C₁₋ₑ₄alkylS(CH₃)₂⁺, and C₁₋₄alkylC(O)NH₂; preferably R⁸ is hydroxy C₁₋₄alkyl, C₁₋₄alkylNHR⁹, C₁₋₆alkylC(O)OH, C₁₋₄alkylNHC(NH)NH₂, hydrogen, C₁₋₄alkyl, aminoC₁₋₆alkyl, —CH(OH)CH₃, —(O)OH, C₁₋₄alkyl(CO₂H)₂, —SO₃H, C₁₋₄alkylS(O)CH₃, C₁₋ₑ₄alkylS(CH₃)₂⁺, and C₁₋₄alkylC(O)NH₂; preferably R⁸ is —CH₂OH, —CH₂NHR³, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂.

Aspect 10. According to particular embodiments, the present invention provides a labeling precursor in the form of a compound of formula (I),
wherein,
each Z¹ is independently selected from the group consisting of —OR¹¹, —C(O)R¹¹, nitro, hydroxyl, C₁₋₆alkyl, aryl, heteroaryl, —SR¹¹, —NR¹²C(O)R¹³, —C(O)₂R¹², cyano, —S(O)₂R¹⁰, halo, haloC₁₋₆alkyl, haloC₁₋₆alkyloxy, heterocyclyl, amino, —NR¹¹R¹², —C(O)NR¹²R¹³, —S(O)R¹⁰, —S(O)₂NR¹²R¹³; preferably Z¹ is C₁₋₆alkyloxy, arylC₁₋₆alkyloxy, aryloxy, heteroaryloxy, heterocyclyloxy, C₁₋₆alkylcarbonyl, arylC₁₋₆alkylcarbonyl, arylcarbonyl, nitro, hydroxyl, C₁₋₆alkyl, aryl, heteroaryl, C₁₋₆alkylthio, arylC₁₋₆alkylthio, arylthio, C₁₋₆alkylcarbonylamino, arylC₁₋₆alkylcarbonylamino, arylcarbonylamino, hydroxycarbonyl, C₁₋₆alkyloxycarbonyl, arylC₁₋₆alkyloxycarbonyl, aryloxycarbonyl, cyano, C₁₋₆alkylsulfonyl, arylC₁₋₆alkylsulfonyl, arylsulfonyl, halo, haloC₁₋₆alkyl, haloC₁₋₆alkyloxy, heterocyclyl, amino, mono-C₁₋₆alkylamino, di-C₁₋₆alkylamino, mono-arylamino, di-arylamino: preferably Z¹ is C₁₋₆alkyloxy, aryloxy, C₁₋₆alkylcarbonyl, arylcarbonyl, nitro, hydroxyl, C₁₋₆alkyl, aryl, heteroaryl, C₁₋₆alkylthio, arylthio, C₁₋₆alkylcarbonylamino, arylC₁₋₆alkylcarbonylamino, arylcarbonylamino, hydroxycarbonyl, C₁₋₆alkyloxycarbonyl, aryloxycarbonyl, cyano, C₁₋₆alkylsulfonyl arylsulfonyl, halo, haloC₁₋₆alkyl, haloC₁₋₆alkyloxy, heterocyclyl, amino, mono-C₁₋₆alkylamino, di-C₁₋₆alkylamino: preferably Z¹ is C₁₋₄alkyloxy, aryloxy, C₁₋₄alkylcarbonyl, arylcarbonyl, nitro, hydroxyl, C₁₋₄alkyl, aryl, heteroaryl, C₁₋₄alkylthio, arylthio, C₁₋₄alkylcarbonylamino, arylC₁₋₄alkylcarbonylamino, arylcarbonylamino, hydroxycarbonyl, C₁₋₆alkyloxycarbonyl, aryloxycarbonyl, cyano, C₁₋₄alkylsulfonyl arylsulfonyl, haloC₁₋₄alkyl, mono-C₁₋₄alkylamino, di-C₁₋₄alkylamino;
wherein said C₁₋₆alkyl, or aryl, can be unsubstituted or substituted with one or more C₁₋₄alkyl, methoxy, nitro, —C(O)aryl, halo, trifluoromethyl, trifluoromethoxy: preferably said groups can be unsubstituted or substituted with one, two or three C₁₋₄alkyl, methoxy, nitro, —C(O)aryl, halo, trifluoromethyl, trifluoromethoxy.

Aspect 11. A solvate, hydrate, salt or prodrug of the compound of any one of aspects 1 to 10.

Aspect 12. Another aspect of the present invention provides a metal complex of a compound as defined herein (also called labeling precursor) such as the ones defined in any one of aspects 1 to 11, and an element of Group VII of the Periodic Table. Preferably, said element is a radionuclide, more preferably the element is ^{99m}Tc or ¹⁸⁸Re or ¹⁸⁶Re.

Aspect 13. Yet another aspect of the present invention relates to a pharmaceutical composition comprising one or more pharmaceutically acceptable excipients and a metal complex as defined in the previous aspects.

Aspect 14. According to another aspect, the present invention also encompasses a metal complex as defined hereinabove or a pharmaceutical composition as defined hereinabove, for use as a medicament.

Aspect 15. According to yet another aspect, the present invention also encompasses a metal complex according to aspect 12 or a pharmaceutical composition according to aspect 13, for use in the treatment or prevention of cancer. In a preferred embodiment of said aspect, the radionuclide used for therapeutic use is ¹⁸⁸Re or ¹⁸⁶Re.

Aspect 16. According to yet another aspect, the present invention also encompasses a metal complex according to aspect 12, or a pharmaceutical composition according to aspect 13, for use as a radiodiagnostic agent for use in in-vivo imaging or detection of tumor or cancer cells or of in-vivo diagnosis of cancer in a subject. Preferred imaging methods are: positron emission tomography (PET), PET computed tomography (PET-CT) or single-photon emission tomography (SPECT). In a preferred embodiment of said aspect, the radionuclide used for imaging is ^{99m}Tc.

Aspect 17. The metal complex or pharmaceutical composition for use according to aspect 15 or 16, wherein said cancer is a PSMA-expressing cancer or tumor.

Aspect 18. The metal complex or pharmaceutical composition for use according to aspect 17, wherein said cancer is selected from the group consisting of: conventional renal cell cancer, transitional cell of the bladder cancer, non-small-cell lung cancer, testicular-embryonal cancer, neuroendocrine cancer, colon cancer, prostate cancer, and breast cancer, preferably prostate cancer.

Aspect 19. A method of treating or preventing cancer in a subject comprising administering a therapeutically effective amount of the metal complex according to aspect 12, or a pharmaceutical composition according to aspect 13 to said patient. In a preferred embodiment of said aspect, the radionuclide used for therapeutic use is ¹⁸⁸Re or ¹⁸⁶Re.

Aspect 20. A method of in-vivo imaging or detection of tumor or cancer cells or of in-vivo diagnosis of cancer in a subject, comprising administering a suitable amount of the metal complex according to aspect 12, or a pharmaceutical composition according to aspect 13 to said patient and visualizing said metal complex using an in-vivo radio-imaging method. Preferred imaging methods are: positron emission tomography (PET), PET computed tomography (PET-CT) or single-photon emission tomography (SPECT). In a preferred embodiment of said aspect, the radionuclide used for imaging is ^{99m}Tc.

Aspect 21. The method according to aspect 19 or 20, wherein said cancer is a PSMA-expressing cancer or tumor, more preferably wherein said cancer is selected from the group consisting of: conventional renal cell cancer, transitional cell of the bladder cancer, non-small-cell lung cancer, testicular-embryonal cancer, neuroendocrine cancer, colon cancer, prostate cancer, and breast cancer, preferably prostate cancer.

Aspect 22. The present invention also encompasses a radiolabelling kit comprising:
the labelling precursor (or compound) as defined hereinabove,
a suitable buffering system, preferably selected from the group consisting of: phosphate buffers, acetate buffers, formate buffers, and HEPES buffers, more preferably phosphate buffers, even more preferably a sodium-phosphate buffer; and
a suitable reducing agent, enabling the reduction of the pertechnetate/perrhenate to a suitable oxidation state for coordination, most likely: Tc(V)O/Re(V)O, such as but not limited to: ascorbic acid, sodium borohydride, sodium dithionite, phosphines such as TCEP, and stannous chloride (Tin(II)chloride), preferably stannous chloride most preferably stannous chloride (tin(II)chloride).

Aspect 23. The radiolabelling kit according to aspect 22, wherein said compound and buffer are present in one or more vials, preferably glass vials, more preferably siliconized vials such as borosilicate glass vials.

Aspect 24. The radiolabelling kit according to aspect 22 or 23, wherein said compound and/or buffer are present in lyophilised form.

Aspect 25. In some embodiments of the kit of any one of aspects 22 to 24, the reducing agents are selected from the group consisting of: ascorbic acid, sodium borohydride, sodium dithionite, phosphines such as TCEP, and stannous chloride (Tin(II)chloride), preferably stannous chloride.

Aspect 26. The radiolabelling kit according to any one of aspects 22 to 25, wherein said kit also comprises a suitable anti-oxidant agent such as but not limited to: sodium ascorbate/ascorbic acid mixtures, sodium borohydride, sodium dithionite, and stannous chloride.

Aspect 27. The radiolabelling kit according to any one of aspects 22 to 26, wherein said kit also comprises a suitable auxillary agents or ligands enabling the protection against reoxidation of Tc(V)O/Re(V)O as competing reaction to coordination, such as but not limited to: tartrate, citrate or glucoheptonate.

Aspect 28. Additionally sequestering agents competing with the chelator for radiometal impurities can be present as well in the kit. Preferably, such sequestering agents are selected from:, mono-, di-, oligo-, or polysaccharides, polynucleate agents, glucoheptonate, tartrate salts and citrate salts.

Aspect 29. In some embodiments, the kit according to any one of aspects 22 to 28 can also include a stabilizer enabling the storage of the kit known in the art, and/or further excipients such as lyophilization agents, matrix reagents or solubilizers known in the art.

Aspect 30. In yet another embodiment, the present invention also provides for a method of radiolabelling a compound or labelling precursor according to any one of aspects 1 to 11, comprising the steps of:
providing a compound or labelling precursor according to any one of aspects 1 to 11,
providing a suitable buffering system
providing a radionuclide, preferably selected from ^{99m}Tc or ¹⁸⁸Re and ¹⁸⁶Re
providing a suitable reducing agent
mixing all components at a suitable pH and allowing the complexation of the radionuclide and labelling precursor to occur, thereby obtaining a radiolabelled compound.

Aspect 31. The method of aspect 30, wherein said buffering system is selected from the group consisting of: phosphate buffers, acetate buffers, formate buffers, and HEPES buffers, more preferably phosphate buffers, even more preferably a sodium-phosphate buffer.

Aspect 32. The method of aspects 30 or 31, wherein when the radionuclide used is ^{99m}Tc, the precursor and buffer are mixed and a suitable amount of pertechnetate is eluated in saline from a molybdenum-99 (⁹⁹Mo/⁹⁹Tc) generator into said mixture. Preferably, the pH of said mixture is set at between 2 and 12 preferably between 7 to 10, and preferably, the temperature of the mixture is kept between 20 and 130° C., preferably from about 20 to 98° C. for about 2 to 60 minutes preferably 5 to 15 minutes.

Aspect 32. The method of any one of aspects 30 to 31, wherein when the radionuclide used is ¹⁸⁸Re, the precursor and buffer are mixed and a suitable amount of Rhenium is eluted in saline from a tungsten-188 (¹⁸⁸W/¹⁸⁸Re) generator into said mixture (with or without postprocessing of the generator eluate). Preferably, the pH is set at between 2 and 9 and the mixture is heated at about 95 to 99° C. for about 5 to 60 minutes.

Aspect 33. The method of any one of aspects 30 to 32, wherein when the radionuclide used is ¹⁸⁶Re, the precursor and buffer are mixed and a suitable amount of Rhenium-186 is produced from a cyclotron or reactor and added into said mixture. Preferably, the pH is set at between 2 and 12, preferably between 2 and 9 and the temperature of the mixture is kept between 20 and 130° C., preferably from about 20 to 98° C. for about 5 to 60 minutes.

Aspect 34. Use of the metal complex according to aspect 12, or a pharmaceutical composition according to aspect 13 for the manufacturing of a medicament for treating or preventing cancer in a subject. In a preferred embodiment of said aspect, the radionuclide used for therapeutic use is ¹⁸⁸Re or ¹⁸⁶Re.

Aspect 35. Use of the metal complex according to aspect 12, or a pharmaceutical composition according to aspect 13 for the manufacturing of a medicament for in-vivo imaging or detection of tumor or cancer cells or of in-vivo diagnosis of cancer in a subject. Preferred imaging methods are: positron emission tomography (PET), PET computed tomography (PET-CT) or single-photon emission tomography (SPECT). In a preferred embodiment of said aspect, the radionuclide used for imaging is ^{99m}Tc.

Aspect 36. The method according to aspect 34 or 35, wherein said cancer is a PSMA-expressing cancer or tumor, more preferably wherein said cancer is selected from the group consisting of: conventional renal cell cancer, transitional cell of the bladder cancer, non-small-cell lung cancer, testicular-embryonal cancer, neuroendocrine cancer, colon cancer, prostate cancer, and breast cancer, preferably prostate cancer.

The above and further aspects and preferred embodiments of the invention are described in the following sections and in the appended claims. The subject matter of the appended claims is hereby specifically incorporated in this specification.

### BRIEF DESCRIPTION OF THE FIGURES

The following description of the figures of specific embodiments of the invention is merely exemplary in nature and is not intended to limit the present teachings, their application or uses.

FIG. 1 represents photographs obtained using planar imaging technique. The planar imaging photographs show LNCaP tumor bearing mice that have been injected with exemplary compounds according to the invention. An activity standard (approx. 1 MBq of the respective tracer, indicated with an arrow) was placed next to the mice as a control of the imaging.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . , to . . . ” or the expression “between . . , and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc, of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

When describing the present invention, the terms used are to be construed in accordance with the following definitions, unless a context dictates otherwise.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e. a compound that is sufficiently robust to survive isolation from a reaction mixture.

Where groups can be substituted, such groups may be substituted with one or more, and preferably one, two or three substituents. Preferred substituents may be selected from but not limited to, for example, the group comprising halo, hydroxyl, alkyl, alkoxy, trifluoromethyl, trifluoromethoxy, cycloalkyl, aryl, arylalkyl, heterocyclyl, heteroaryl, cyano, amino, nitro, carboxyl, and mono- or dialkylamino.

The term “halo” or “halogen” as a group or part of a group is generic for fluoro, chloro, bromo, iodo.

The term “hydroxyl” or “hydroxy” as used herein refers to the group —OH.

The term “cyano” as used herein refers to the group —C≡N.

The term “amino” as used herein refers to the —NH₂ group.

The term “nitro” as used herein refers to the —NO₂ group.

The term “carboxy” or “carboxyl” or “hydroxycarbonyl” as used herein refers to the group —CO₂H.

The term “aminocarbonyl” as used herein refers to the group —CONH₂.

The term “C₁₋₆alkyl”, as a group or part of a group, refers to a hydrocarbyl group of comprising from 1 to 6 carbon atoms. C₁₋₆alkyl groups may be linear or branched and may be substituted as indicated herein. Generally, alkyl groups of this invention comprise from 1 to 6 carbon atoms, preferably from 1 to 5 carbon atoms, preferably from 1 to 4 carbon atoms, more preferably from 1 to 3 carbon atoms, still more preferably 1 to 2 carbon atoms. When a subscript is used herein following a carbon atom, the subscript refers to the number of carbon atoms that the named group may contain. For example, “C₁₋₆alkyl” includes methyl, ethyl, n-propyl, i-propyl, butyl and its isomers (e.g. n-butyl, i-butyl and t-butyl); pentyl and its isomers, hexyl and its isomers. For example, “C₁₋₅alkyl” includes all linear or branched alkyl groups with between 1 and 5 carbon atoms, and thus includes methyl, ethyl, n-propyl, i-propyl, butyl and its isomers (e.g. n-butyl, i-butyl and t-butyl); pentyl and its isomers. For example, “C₁₋₄alkyl” includes all linear or branched alkyl groups with between 1 and 4 carbon atoms, and thus includes methyl, ethyl, n-propyl, i-propyl, butyl and its isomers (e.g. n-butyl, i-butyl and t-butyl). For example “C₁₋₃alkyl” includes all linear or branched alkyl groups with between 1 and 3 carbon atoms, and thus includes methyl, ethyl, n-propyl, i-propyl.

When the term “C₁₋₆alkyl” is used as a suffix following another term, as in “hydroxy C₁₋₆alkyl alkyl,” this is intended to refer to an C₁₋₆alkyl group, as defined above, being substituted with one or two (preferably one) substituent(s) selected from the other, specifically-named group, also as defined herein. The term “hydroxyC₁₋₆alkyl” therefore refers to a —R^{a}—OH group wherein R^{a} is alkylene as defined herein.

The term “haloC₁₋₆alkyl” as a group or part of a group, refers to a C₁₋₆alkyl group having the meaning as defined above wherein one, two, or three hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl, trichloromethyl, tribromomethyl, and the like.

The term “trifluoromethyl” as used herein refers to the group —CF₃.

The term “trifluoromethoxy” as used herein refers to the group —OCF₃.

The term “C₁₋₆alkyloxy” or “C₁₋₆alkyloxy”, as a group or part of a group, refers to a group having the formula —OR^{b} wherein R^{b} is C₁₋₆alkyl as defined herein above. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy.

The term “haloC₁₋₆alkyloxy” as a group or part of a group, refers to a C₁₋₆alkyloxy group having the meaning as defined above wherein one, two, or three hydrogen atoms are each replaced with a halogen as defined herein. Non-limiting examples of such haloC₁₋₆alkyl groups include chloromethyloxy, 1-bromoethyloxy, fluoromethyloxy, difluoromethyloxy, trifluoromethyloxy, 1,1,1-trifluoroethyloxy, trichloromethyloxy, tribromomethyloxy, and the like.

The term “C₂₋₆alkenyl” as a group or part of a group, refers to an unsaturated hydrocarbyl group, which may be linear, or branched comprising one or more carbon-carbon double bonds and comprising from 2 to 6 carbon atoms. For example, C₂₋₄alkenyl includes all linear, or branched alkenyl groups having 2 to 4 carbon atoms. Examples of C₂₋₆alkenyl groups are ethenyl, 2-propenyl, 2-butenyl, 3-butenyl, 2-pentenyl and its isomers, 2-hexenyl and its isomers, 2,4-pentadienyl, and the like.

The term “aryl”, as a group or part of a group, refers to a polyunsaturated, aromatic hydrocarbyl group having a single ring (i.e. phenyl) or multiple aromatic rings fused together (e.g. naphthyl), or linked covalently, typically comprising 6 to 12 carbon atoms: wherein at least one ring is aromatic, preferably comprising 6 to 10 carbon atoms, wherein at least one ring is aromatic. The aromatic ring may optionally include one to two additional rings (either cycloalkyl, heterocyclyl or heteroaryl) fused thereto. Examples of suitable aryl include C₆₋₁₂aryl, preferably C₆₋₁₀aryl, more preferably C₆₋₈aryl. Non-limiting examples of aryl comprise phenyl, biphenylyl, biphenylenyl, or 1-or 2-naphthanelyl; 5- or 6-tetralinyl, 1-, 2-, 3-, 4-, 5-, 6-, 7- or 8-azulenyl, 4-, 5-, 6 or 7-indenyl, 4- or 5-indanyl, 5-, 6-, 7- or 8-tetrahydronaphthyl, 1,2,3,4-tetrahydronaphthyl, 1,4-dihydronaphthyl; anthracenyl, dibenzosuberyl, and 1-, 2-, 3-, 4- or 5-pyrenyl. A “substituted aryl” refers to an aryl group having one or more substituent(s) (for example 1, 2 or 3 substituent(s), or 1 to 2 substituent(s)), at any available point of attachment.

The term “aryloxy”, as a group or part of a group, refers to a group having the formula —OR^{g} wherein R^{g} is aryl as defined herein above.

The term “arylC₁₋₆alkyl”, as a group or part of a group, means a C₁₋₆alkyl as defined herein, wherein at least one hydrogen atom is replaced by at least one aryl as defined herein. Non-limiting examples of arylC₁₋₆alkyl group include benzyl, phenethyl, dibenzylmethyl, methylphenylmethyl, 3-(2-naphthyl)-butyl, 9-anthrylmethyl. 9-fluorenymethyl and the like.

The term “arylC₁₋₆alkyloxy”, as a group or part of a group, refers to a group having the formula —OR^{g} wherein R^{g} is arylC₁₋₆alkylas defined herein above.

The terms “heterocyclyl” or “heterocycloakyl” or “heterocyclo”, as a group or part of a group, refer to non-aromatic, fully saturated or partially unsaturated cyclic groups (for example, 3 to 7 member monocyclic, 7 to 11 member bicyclic, or comprising a total of 3 to 10 ring atoms) which have at least one heteroatom in at least one carbon atom-containing ring; wherein said ring may be fused to an aryl, cycloalkyl, heteroaryl or heterocyclyl ring. Each ring of the heterocyclyl group containing a heteroatom may have 1, 2, 3 or 4 heteroatoms selected from N, O and/or S, where the N and S heteroatoms may optionally be oxidized and the N heteroatoms may optionally be quaternized, and wherein at least one carbon atom of heterocyclyl can be oxidized to form at least one C═O. The heterocyclic group may be attached at any heteroatom or carbon atom of the ring or ring system, where valence allows. The rings of multi-ring heterocycles may be fused, bridged and/or joined through one or more spiro atoms.

Non limiting exemplary heterocyclic groups include xanthenyl, aziridinyl, oxiranyl, thiiranyl, piperidinyl, azetidinyl, oxetanyl, pyrrolidinyl, thietanyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, chromanyl (also known as 3,4-dihydrobenzo[b]pyranyl), isoindolinyl, 2H-pyrrolyl, 1-pyrrolinyl, 2-pyrrolinyl, 3-pyrrolinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopipcridinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxctan-2-yl and oxctan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “chromanyl” as used herein includes chroman-2-yl, chroman-3-yl, chroman-4-yl, chroman-5-yl, chroman-6-yl, chroman-7-yl and chroman-8-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used hercin includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl -3-yl and tetrahydrothiophenyl -4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used hercin includes 1,2,3-trioxan-4-yl, 1,2,3-trioxay-5-yl, 1,2,4-trioxay-3-yl, 1,2,4-trioxay-5-yl, 1,2,4-trioxay-6-yl and 1,3,4-trioxay-2-yl. The term “azepanyl” as used herein includes azepan-1-yl, azepan-2-yl, azepan-1-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

The term “heterocyclyloxy”, as a group or part of a group, refers to a group having the formula —O—Rⁱ wherein Rⁱ is heterocyclyl as defined herein above.

The term “heterocyclylC₁₋₆alkyl”, as a group or part of a group, means a C₁₋₆alkyl as defined herein, wherein at least one hydrogen atom is replaced by at least one heterocyclyl as defined herein.

The term “heteroaryl” as a group or part of a group, refers but is not limited to 5 to 12 carbon-atom aromatic rings or ring systems containing 1 or 2 rings which can be fused together or linked covalently. typically containing 5 to 6 atoms; at least one of which is aromatic in which one or more carbon atoms in one or more of these rings can be replaced by N, O and/or S atoms where the N and S heteroatoms may optionally be oxidized and the N heteroatoms may optionally be quaternized, and wherein at least one carbon atom of said heteroaryl can be oxidized to form at least one C═O. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl: preferably said heteroaryl group is selected from the group consisting of pyridyl, 1,3-benzodioxolyl, benzo[d]oxazol-2(3H)-one, 2,3-dihydro-benzofuranyl, pyrazinyl, pyrazolyl, pyrrolyl, isoxazolyl, thiophenyl, imidazolyl, benzimidazolyl, pyrimidinyl, triazolyl and thiazolyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl, “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazoi-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl, -indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2, 1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

The term “heteroaryloxy”, as a group or part of a group, refers to a group having the formula —O—R^{k} wherein R^{k} is heteroaryl as defined herein above.

The term “heteroarylC₁₋₆alkyl”, as a group or part of a group, means a C₁₋₆alkyl as defined herein, wherein at least one hydrogen atom is replaced by at least one heteroaryl as defined herein. Non limiting examples of heteroarylC₁₋₆alkyl are 2-quinolinylmethyl, 2-(4-pyridyl)-ethyl, and the like.

The term “C₁₋₆alkylthioC₁₋₆alkylene”, as a group or part of a group, refers to a group of formula —R^{a}—S—R^{b} wherein R^{a} is C₁₋₆alkylene as defined herein, and b^{a} is C₁₋₆alkyl as defined herein.

The term “mercaptoC₁₋₆alkyl” or “C₁₋₆alkylthio”, as a group or part of a group, refers to a group of formula —SR^{a} wherein R^{a} is C₁₋₆alkyl as defined herein.

The term “arylthio”, as a group or part of a group, refers to a group of formula —SR^{a} wherein R^{a} is aryl as defined herein.

The term “C₁₋₆alkyarylthio”, as a group or part of a group, refers to a group of formula —SR^{a} wherein R^{a} is C₁₋₆alkylaryl as defined herein.

The term “aminoC₁₋₆alkyl”, as a group or part of a group, refers to a group of formula —R^{a}—NR^{o}R^{p} wherein R^{a} is C₁₋₆alkylene, R^{o} is hydrogen or C₁₋₆alkyl as defined herein, and R^{p} is hydrogen or C₁₋₆alkyl as defined herein.

The term “mono- or di-C₁₋₆alkylamino”, as a group or part of a group, refers to a group of formula —N(R^{o})(R^{p}) wherein R^{o} and R^{p} are each independently selected from hydrogen, or C₁₋₆alkyl, wherein at least one of R^{o} or R^{p} is C₁₋₆alkyl. Thus, C₁₋₆alkylamino include mono-alkyl amino group (e.g. mono-C₁₋₆alkylamino group such as methylamino and ethylamino), and di-alkylamino group (e.g. di-C₁₋₆alkylamino group such as dimethylamino and diethylamino). Non-limiting examples of suitable mono- or di-C₁₋₆alkylamino groups include n-propylamino, isopropylamino, n-butylamino, i-butylamino, sec-butylamino, 1-butylamino, pentylamino, n-hexylamino, di-n-propylamino, di-i-propylamino, ethylmethylamino, methyl-n-propylamino, methyl-i-propylamino, n-butylmethylamino, i-butylmethylamino, 1-butylmethylamino, ethyl-n-propylamino, ethyl-i-propylamino, n-butylethylamino, i-butylethylamino. 1-butylethylamino, di-n-buty lamino, di-i-butylamino, methylpentylamino, methylhexylamino, ethylpentylamino, ethylhexylamino, propylpentylamino, propylhexylamino, and the like.

The term “mono- or di-arylamino”, as a group or part of a group, refers to a group of formula —N(R^{q})(R^{r}) wherein R^{q} and R^{r} are each independently selected from hydrogen, aryl, or alkyl, wherein at least one of R^{q} or R^{r} is aryl.

The term “alkylcarbonyl”, as a group or part of a group, refers to a group of formula —CO—R^{b}, wherein R^{b} is alkyl as defined herein.

The term “arylC₁₋₆alkylcarbonyl”, as a group or part of a group, refers to a group of formula —CO—R^{b}, wherein R^{b} is arylC₁₋₆alkyl as defined herein.

The term “arylcarbonyl”, as a group or part of a group, refers to a group of formula —CO—R^{b}, wherein R^{b} is aryl as defined herein.

The term “C₁₋₆alkyloxycarbonyl”, as a group or part of a group, refers to a group of formula —COO—R^{b}, wherein R^{b} is C₁₋₆alkyl as defined herein.

The term “arylC₁₋₆alkyloxycarbonyl”, as a group or part of a group, refers to a group of formula —COO—R^{b}, wherein R^{b} is arylC₁₋₆alkyl as defined herein.

The term “aryloxycarbonyl”, as a group or part of a group, refers to a group of formula —COO—R^{b}, wherein R^{b} is aryl as defined herein.

The term “C₁₋₆alkylcarbonylamino”, as a group or part of a group, refers to a group of formula —NR^{o}—CO—R^{b}, wherein R^{o} is selected from hydrogen, or C₁₋₆alkyl and R^{b} is C₁₋₆alkyl as defined herein.

The term “arylC₁₋₆alkylcarbonylamino”, as a group or part of a group, refers to a group of formula —NR^{o}—CO—R^{b}, wherein R^{o} is selected from hydrogen, or arylC₁₋₆alkyl and R^{b} is arylC₁₋₆alkyl as defined herein.

The term “alrylcarbonylamino”, as a group or part of a group, refers to a group of formula —NR^{o}—CO—R^{b}, wherein R^{o} is selected from hydrogen, or aryl and R^{b} is aryl as defined herein.

The term “C₁₋₆alkylsulfonyl”, as a group or part of a group, refers to a group of formula —S(O)₂—R^{b}, wherein R^{b} is C₁₋₆alkyl as defined herein.

The term “arylC₁₋₆alkylsulfonyl”, as a group or part of a group, refers to a group of formula —S(O)₂—R^{b}, wherein R^{b} is arylC₁₋₆alkyl as defined herein.

The term “arylsulfonyl”, as a group or part of a group, refers to a group of formula —S(O)₂—R^{b}, wherein R^{b} is aryl as defined herein.

The term “mono- or diC₁₋₆alkylaminocarbonyl”, as a group or part of a group, refers to a group of formula —CONR^{o}R^{p} wherein R^{o}R^{p} are each independently selected from hydrogen, or C₁₋₆alkyl, wherein at least one of R^{o} or R^{p} is C₁₋₆alkyl.

The term “mono- or diarylC₁₋₆alkylaminocarbonyl”, as a group or part of a group, refers to a group of formula —CONR^{o}R^{p} wherein R^{o}R^{p} are each independently selected from hydrogen, or arylC₁₋₆alkyl, wherein at least one of R^{o} or R^{p} is arylC₁₋₆alkyl.

The term “mono- or diarylaminocarbonyl”, as a group or part of a group, refers to a group of formula —CONR^{o}R^{p} wherein R^{o}R^{p} are each independently selected from hydrogen, or aryl, wherein at least one of R^{o} or R^{p} is aryl.

Whenever used in the present invention the term “compounds of the invention” or a similar term is meant to include the compounds of general formula (I), (IA), (IB), (IC) or (ID) and any subgroup thereof. This term also refers to the compounds as depicted in Table 2 and their derivatives, N-oxides, salts, solvates, hydrates, tautomeric forms, analogues, pro-drugs, esters and metabolites, as well as their quaternized nitrogen analogues. The N-oxide forms of said compounds are meant to comprise compounds wherein one or several nitrogen atoms are oxidized to the so-called N-oxide.

As used herein and unless otherwise stated, the term “stereoisomer” refers to all possible different isomeric as well as conformational forms which the compounds of structural formula herein may possess, in particular all possible stercochemically and conformationally isomeric forms, all diastereomers, enantiomers and/or conformers of the basic molecular structure. Some compounds of the present invention may exist in different tautomeric forms, all of the latter being included within the scope of the present invention.

The present invention includes all possible stereoisomers compounds of formula (I) and any subgroup thereof. When a compound is desired as a single enantiomer, such may be obtained by stercospecific synthesis, by resolution of the final product or any convenient intermediate, or by chiral chromatographic methods as each are known in the art. Resolution of the final product, an intermediate, or a starting material may be effected by any suitable method known in the art. See, for example, Stercochemistry of Organic Compounds by E. L. Eliel, S. H. Wilen, and L. N. Mander (Wiley-Interscience, 1994), incorporated by reference with regard to stereochemistry. A structural isomer is a type of isomer in which molecules with the same molecular formula have different bonding patterns and atomic organization. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety.

The compounds of the invention may be in the form of salts, preferably pharmaceutically acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of the compounds of formula (I) and any subgroup thereof include the acid addition and base salts thereof. Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, glucoptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine. benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The term “prodrug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Prodrugs of the compounds of the invention can be prepared by modifying functional groups present in said component in such a way that the modifications are cleaved, either in routine manipulation or in vivo, to the parent component. Typical examples of prodrugs are described for instance in WO 99/33795, WO 99/33815, WO 99/33793 and WO 99/33792 all incorporated herein by reference. Prodrugs are characterized by increased bio-availability and are readily metabolized into the active inhibitors in vivo. The term “prodrug”, as used herein, means any compound that will be modified to form a drug species, wherein the modification may take place either inside or outside of the body, and either before or after the pre-drug reaches the area of the body where administration of the drug is indicated.

As used herein, an “element of Group VII of the Periodic Table” corresponds to transition metals manganese (Mn), technetium (Tc), rhenium (Rc) and bohrium (Bh).

The term “radionuclide” is to be interpreted in the broad commonly used definition in the art, and thus refers to any atom that contains excess nuclear energy that renders said atom unstable. More particularly the term refers to an isotope of natural or artificial origin which shows radioactive properties. Non-limiting examples of radionuclide include ^{99m}Tc or ¹⁸⁸Re or ¹⁸⁶Re.

The term “labeled” as used herein means “radiolabeled” and is more precisely directed to a compound comprising or complexed with at least one radionuclide.

It is understood that when reference is made herein to “prostate-specific membrane antigen”, abbreviated herein and in the art as “PSM” and “PSMA” and interchangeably annotated in the art by the non-limiting synonyms “glutamate carboxypeptidase 2” (abbreviation: “GCP2”), “glutamate carboxypeptidase II” (“GCPII”), “cell growth-inhibiting gene 27 protein”, “folate hydrolase 1”, “folylpoly-gamma-glutamate carboxypeptidase” (“FGCP”), “membrane glutamate carboxypeptidase” (“mGCP”), “N-acetylated-alpha-linked acidic dipeptidase I” (“NAALADase I”), NAAG peptidase, and “pteroylpoly-gamma-glutamate carboxypeptidase” that reference is made to the protein having as UniProt identifier (www.uniprot.org) Q04609 (FOLH1_HUMAN) and NCBI reference (ncbi.nlm.nih.gov) NP_004467.1 as encoded in Homo sapiens by the gene FOLH1 unless specified otherwise. PSMA is a zinc metalloenzyme that is categorised as a class II membrane glycoprotein that catalises the hydrolysis of hydrolysis of N-acetylaspartylglutamate (NAAG) to glutamate and N-acetylaspartate (NAA). The canonical sequence of PSMA is by means of example reproduced below (SEQ ID NO: 1):

MWNLLHETDSAVATARRPRWLCAGALVLAGGFFLLGELFGWFIKSSNEA
TNITPKHNMKAFLDELKAENIKKFLYNFTQIPHLAGTEQNFQLAKQIQS
QWKEFGLDSVELAHYDVLLSYPNKTHPNYISIINEDGNEIFNTSLFEPP
PPGYENVSDIVPPFSAFSPQGMPEGDLVYVNYARTEDFFKLERDMKINC
SGKIVIARYGKVFRGNKVKNAQLAGAKGVILYSDPADYFAPGVKSYPDG
WNLPGGGVQRGNILNLNGAGDPLTPGYPANEYAYRRGIAEAVGLPSIPV
HPIGYYDAQKLLEKMGGSAPPDSSWRGSLKVPYNVGPGFTGNFSTQKVK
MHIHSTNEVTRIYNVIGTLRGAVEPDRYVILGGHRDSWVFGGIDPQSGA
AVVHEIVRSFGTLKKEGWRPRRTILFASWDAEEFGLLGSTEWAEENSRL
LQERGVAYINADSSIEGNYTLRVDCTPLMYSLVHNLTKELKSPDEGFEG
KSLYESWTKKSPSPEFSGMPRISKLGSGNDFEVFFQRLGIASGRARYTK
NWETNKFSGYPLYHSVYETYELVEKFYDPMFKYHLTVAQVRGGMVFELA
NSIVLPFDCRDYAVVLRKYADKIYSISMKHPQEMKTYSVSFDSLESAVK
NFTEIASKESERLQDFDKSNPIVLRMMNDQLMFLERAFIDPLGLPDRPF
YRHVIYAPSSHNKYAGESFPGIYDALFDIESKVDPSKAWGEVKRQIYVA
AFTVQAAAETLSEVA

While SEQ ID NO: 1 as depicted above is generally accepted as the canonical sequence of PSMA, it is evident that the term “PSMA” and synonyms thereof equally encompass any isoforms of PSMA, truncated versions of PSMA, and genetic polymorphisms of PSMA. In particular, the term “PSMA” is intended to cover any protein sequence having at least 80%, preferably at least 85%, more preferably at least 90%, more preferably at least 95%, yet more preferably at least 97.5%, most preferably at least 99% sequence identity to SEQ ID NO: 1. A skilled person further appreciates that the radiotheranostics described in the present invention are capable of selectively binding any protein that contains at least the extracellular active center of PSMA.

Related hereto, a person skilled in the art is well aware of methods and tools to verify sequence homology, sequence similarity or sequence identity between different sequences of amino acids or nucleic acids. Non-limiting examples of such methods and tools are Protein BLAST (https://blast.ncbi.nlm.nih.gov/Blast.cgi), ClustalW2 (https://www.ebi.ac.uk/Tools/msa/clustalw2/), SIM alignment tool (https://web.cxpasy.org/sim/), TranslatorX (http://translatorx.co.uk/) and T-COFFEE (https://www.ebi.ac.uk/Tools/msa/tcoffee/). The percentage of identity between two sequences may show minor differences depending on the algorithm choice and parameters.

The term “sequence identity” as used herein refers to the relationship between sequences at the amino acid level. The expression “% identical” is determined by comparing optimally aligned sequences, e.g. two or more, over a comparison window wherein the portion of the sequence in the comparison window may comprise insertions or deletions as compared to the reference sequence for optimal alignment of the sequences. The reference sequence does not comprise insertions or deletions. A reference window is chosen and the “% identity” is then calculated by determining the number of nucleotides (or amino acids) that are identical between the sequences in the window, dividing the number of identical nucleotides (or amino acids) by the number of nucleotides (or amino acids) in the window and multiplying by 100. Unless indicated otherwise, the sequence identity is calculated over the whole length of the reference sequence. A skilled person is aware of the related, yet different interpretations in the art of the terms “similarity”, “homology”, and “identity” (explained in detail in e.g. Pearson, Current protocols in bioinformatics, 2014).

As indicated above, the membrane-bound protease PSMA is overexpressed up to 1000-fold on prostate tumor cells and the precise expression level shows a strong correlation with the disease state, as has been described in the art on numerous occasions (e.g. in Hupe et al., Front Oncol, 2018). Furthermore, PSMA is typically expressed in the neovasculature of several solid tumors such as but not limited to renal carcinoma, breast cancer, non-small-cell lung cancer (NSCLC) and oral cancer. Additionally, PSMA is expressed in a number of healthy tissues such as prostate tissue (in the secretory acinar epithelium), the nervous system (astrocytes and Schwann cells), intestinal tissue (jejunal brush order in the small bowel), kidney (proximal tubes), and salivary glands. A skilled person is aware that expression levels in mainly the kidneys and salivary glands are crucial for determining the dose-limiting factor of radionuclide therapy since these tissues display the highest nontarget uptake in a subject receiving treatment with a PSMA-selective radionuclide. Structurally, the PSMA is characterised by three main domains: an extracellular domain, a transmembrane domain and an enzymatically active extracellular domain which comprises two zinc ions as part of the enzymatic active site. The enzymatic active site of PSMA is composed of two pockets, S1 and S1′. Glutamate-like structures bind to the S1′ pocket which is crucial for high-affinity binding while the S1 pocket is more flexible (Barinka et al., J Med Chem, 2007).

The term “amino acid” encompasses naturally occurring amino acids, naturally encoded amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. Amino acids are referred to herein by either their name, their commonly known three letter symbols or by the one-letter symbols recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). A “non-naturally encoded amino acid” refers to an amino acid that is not one of the 20 common amino acids or pyrrolysine, pyrroline-carboxy-lysine or selenocysteine. The term includes without limitation amino acids that occur by a modification (such as a post-translational modification) of a naturally encoded amino acid, but are not themselves naturally incorporated into a growing polypeptide chain by the translation complex, as exemplified without limitation by N-acetylglucosaminyl-L-serine, N-acetylglucosaminyl-L-threonine, and O-phosphotyrosine. Further examples of non-naturally encoded, un-natural or modified amino acids include 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2,2′-Diaminopimelic acid, 2,3-Diaminopropionic acid, N-Ethylglycine, N-Ethylasparagine, homoserine, homocysteine, Hydroxylysine, allo-Hydroxylysine, 3-Hydroxyproline, 4-Hydroxyproline, Isodesmosine, allo-Isoleucine, N-Methylglycine, N-Methylisoleucine, 6-N-Methyllysine, N-Methylvaline, Norvaline, Norleucine, or Ornithine. Also included are amino acid analogues, in which one or more individual atoms have been replaced either with a different atom, an isotope of the same atom, or with a different functional group. Also included are un-natural amino acids and amino acid analogues described in Ellman et al. Methods Enzymol. 1991. vol. 202. 301-36.

Another aspect of the invention is directed to pharmaceutical compositions comprising one or more pharmaceutically acceptable excipients and a therapeutically effective amount of a metal complex as described herein. In preferred embodiments, the pharmaceutical composition comprises a metal complex comprising Rhenium, preferably Rhenium-188 or 186. In alternative preferred embodiments. the pharmaceutical composition comprises a metal complex comprising Technetium-99m.

In view of the above, any reference to the use of the metal complexes in diagnosis, monitoring, therapy or imaging (or any variation of such language) also encompasses such uses of pharmaceutical compositions comprising the metal complexes described in the present disclosure. The terms “pharmaceutical composition”, “pharmaceutical formulation”, or short “composition” and “formulation” may be used interchangeably and are to be considered synonyms. The pharmaceutical compositions as taught herein may comprise in addition to the one or more pharmaceutically active ingredients, and/or one or more pharmaceutically acceptable carriers (interchangeably referred to as “excipients”. The term “pharmaceutically acceptable” as used herein is consistent with the art and means compatible with the other ingredients of a pharmaceutical composition and not deleterious to the recipient thereof. Suitable pharmaceutical excipients depend on the dosage form and identities of the active ingredients and can be selected by the skilled person (e.g., by reference to Rowe et al., Handbook of Pharmaceutical Excipients 7th Edition 2012). As used herein, the terms “carrier” or “excipient” are used interchangeably and broadly include any and all solvents, diluents, buffers (such as, e.g., neutral buffered saline, phosphate buffered saline, or optionally Tris-HCl, acetate or phosphate buffers), solubilisers (such as, e.g., Tween® 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives (such as, e.g., Thimerosal™, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of such media and agents for the formulation of pharmaceutical compositions is well known in the art. Acceptable diluents and excipients typically do not adversely affect a recipient's homeostasis (e.g., electrolyte balance). The use of such media and agents for pharmaceutical active substances is well known in the art. It is evident that all of the used ingredients should be non-toxic in the concentration contained in the final pharmaceutical composition and should not negatively interfere with the activity of the estetrol component, said estetrol component preferably being present in the pharmaceutical composition as the predominant pharmaceutically active ingredient. In certain embodiments, more than one excipient which a skilled person would classify as belonging to the same group of excipients is added to the pharmaceutical composition. In further embodiments, more than one excipient wherein the different excipients belong to different groups is added to the pharmaceutical composition. In certain embodiments, the excipients may fulfil more than one function and/or be classified by a skilled person as belonging to different groups or classes of excipients. Further illustrative examples of acceptable excipients may include biocompatible, inert or bioabsorbable salts, buffering agents, oligo- or polysaccharides, polymers, viscosity-improving agents. preservatives and the like. Non-limiting exemplary solvents are physiologic saline (0.15 M NaCl, pH 7.0 to 7.4) and 50 mM sodium phosphate, 100 mM sodium chloride. The precise nature of the excipients and solvents will depend on the route of administration. For example, the pharmaceutical composition may be in the form of a parenterally acceptable aqueous solution, which is pyrogen-free and has suitable pH, isotonicity and stability. Preferably, the pH value of the pharmaceutical formulation is in the physiological pH range, such as particularly the pH of the formulation is between about 6 and about 8.5, more preferably between about 6 and about 8.5, even more preferably between about 7 and about 7.5.

While pharmaceutical compositions as intended herein may be formulated for essentially any route of administration, parenteral administration (such as, e.g., subcutaneous, intravenous (I.V.), intramuscular, intraperitoneal or intrasternal injection or infusion) or topical administration may be preferred. The effects attainable can be, for example, systemic, local, tissue-specific, etc., depending of the specific needs of a given application. In certain embodiments, an I.V, bolus injection or infusion may advantageously allow the metal complex to enter circulation and be distributed throughout the body, allowing to label cells and tissues that are characterized by PSMA expression.

One skilled in this art will recognise that the above paragraphs on pharmaceutical compositions are merely illustrative and should by no means be interpreted as being an exhaustive list of embodiments. Indeed, many additional formulations techniques and pharmaceutically-acceptable excipients and solvent solutions are well-known to those skilled in the art, as is the development of suitable dosing and treatment regimens for using the particular compositions described herein in a variety of administration or treatment regimens.

A further aspect of the invention relates to a metal complex as described herein or a pharmaceutical composition as described herein for use as a medicament. The invention further provides in the use of a metal complex as described herein for the manufacture of a medicament for treatment of a disease in a subject.

Further envisaged are methods of treatment and methods of diagnosis which comprise administration of the metal complexes described herein or pharmaceutical compositions described herein to a subject.

Another aspect of the invention relates to a metal complex as described herein or a pharmaceutical composition as described herein for use in the treatment or prevention of cancer. Therefore, also envisaged by the present invention are methods of treating or preventing cancer comprising administration of at least one metal complex as described herein or pharmaceutical composition as described herein. Furthermore, the use of an effective amount of a metal complex as described herein for the manufacture of a medicament for treating or preventing cancer is also intended. In certain embodiments, preventing cancer indicates inhibition of clinical manifestation of cancer. In certain embodiments, the medical use or method of treatment comprises continuous administration of the metal complexes described herein or the pharmaceutical compositions described herein to a subject. In alternative embodiments, the medical use or method of treatment comprises intermittent administration of the metal complexes described herein or the pharmaceutical compositions described herein to a subject.

The terms “treatment” or “treat” are to be interpreted as both the therapeutic treatment of a disease or condition that has already developed, leading to clinical manifestations, such as but not limited to the therapy of an already developed malignancy such as prostate cancer, as well as prophylactic or preventive measures, wherein the goal of the treatment is to prevent, lessen, or reduce the chances of incidence of an undesired clinical affliction, such as to prevent further development and progression of a clinical condition or disease such as prostate cancer. Beneficial or desired clinical results may include, without limitation, alleviation of one or more symptoms, improvement of one or more biological markers, diminishment of extent of disease, stabilized (i.e. not worsening) state of disease, delay or slowing of disease progression, amelioration or palliation of the disease state, slowing tumor growth, reducing the mass of the (main) tumor body, reducing the number and/or size of metastases, and the like. “Treatment” can also mean prolonging survival as compared to expected survival if not receiving treatment, or a reduced risk of mortality.

As used herein, the terms “therapeutic treatment” or “therapy” and the like, refer to treatments wherein the aim is to change a subjects body or a part of a subjects body from an undesired physiological state, disease or disorder which is caused by an infectious agent, to a desired state, such as a less severe state (e.g., amelioration or palliation), or even back to its normal, healthy state (e.g., restoring the health, the physical integrity and the physical well-being of a subject), to keep it (i.e., not worsening) at said undesired physiological status (e.g., stabilization), or slow down progression to a more severe or worse state compared to said undesired physiological change or disorder. Measurable lessening includes any statistically significant decline in a measurable marker or symptom. Statistically significant as used herein refers to p values below 0.05, which is a commonly accepted cutoff score in statistical analysis as a skilled person appreciates. “Treatment” encompasses both curative treatments and treatments directed to reduce symptoms and/or slow progression and/or stabilize the disease.

A skilled person is aware that in order to achieve an effective therapeutic treatment, a therapeutically effective dose needs to be administered to said subject. Therefore, in the context of the present disclosure when reference is made to a metal complex as described herein or a pharmaceutical composition as described herein it is evident that an “effective amount” is envisaged, wherein the “effective amount” refers to an amount necessary to obtain a physiological effect. The physiological effect may be achieved by a single dose or by multiple doses. A “therapeutically effective amount” or “therapeutically effective dose” indicates an amount of metal complex described herein or pharmaceutical composition as described herein that when administered brings about a clinical positive response with respect to treatment of a subject afflicted by a malignancy such as but by no means limited to prostate cancer. A skilled person is aware that terms such as “quantity”, “amount” and “level” are synonyms and have a well-defined meaning in the art and appreciates that these may particularly refer to an absolute quantification of a metal complex as described herein which is considered an effective amount for the applications described herein, or to a relative quantification of the metal complex, such as for example a concentration of metal complex in function of the subject's bodyweight. Suitable values or ranges of values may be obtained from one single subject or from a group of subjects (i.e. at least two subjects). The term “to administer” generally means to dispense or to apply, and typically includes both in vivo administration and ex vivo administration to a tissue, preferably in vivo administration. Generally, compositions may be administered systemically or locally.

For therapeutic applications (e.g. with Rhenium), the preferred dose (activity) is about are 1-10 GBq.

Regarding diagnostic use, (e.g. with Technetium) about 200 to 1000 MBq is typically used for administration. More preferably about 500-800 MBq.

As used herein, the term “cancer” refers to a malignant neoplasm (i.e. a “malignancy”) characterised by deregulated or unregulated cell growth. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). The term “metastatic” or “metastasis” generally refers to the spread of a cancer from one organ or tissue to another non-adjacent organ or tissue. The occurrence of the neoplastic disease in the other non-adjacent organ or tissue is referred to as metastasis. The term “neoplastic disease” generally refers to any disease or disorder characterised by neoplastic cell growth and proliferation, whether benign (not invading surrounding normal tissues, not forming metastases), pre-malignant (pre-cancerous), or malignant (invading adjacent tissues and capable of producing metastases). The term neoplastic disease generally includes all transformed cells and tissues and all cancerous cells and tissues. Neoplastic diseases or disorders include, but are not limited to abnormal cell growth, benign tumors, premalignant or precancerous lesions, malignant tumors, and cancer. As used herein, the terms “tumor” or “tumor tissue” refer to an abnormal mass of tissue that results from excessive cell division. A tumor or tumor tissue comprises tumor cells which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise tumor-associated non-tumor cells, e.g., vascular cells which form blood vessels to supply the tumor or tumor tissue. Non-tumor cells may be induced to replicate and develop by tumor cells, for example, the induction of angiogenesis in a tumor or tumor tissue. Cancers are typically classified into different stages of disease progression in the art. It is envisaged that each of the commonly annotated cancer stages may benefit from treatment with a metal complex as described herein or a pharmaceutical composition as described herein. Furthermore, it is envisaged that the present metal complexes and pharmaceutical compositions described herein are particularly useful for cancers categorized by the TNM staging system as N (node) and M (metastasis) cancer stages (Tio, Gastrointest Endosc, 1996). In certain embodiments, the cancer stage, such as for example the cancer stage of prostate cancer is selected from one or more of the following cancer stages: stage I, stage II, stage IIA, stage IIB, stage IIC, stage III, stage IIIA, stage IIIB, stage IIIC, stage IV, stage IVA, stage IVB. By means of illustration, for prostate cancer these stages correspond to the following clinical images depicted in Table 1:

TABLE 1
prostate cancer stages and associated clinical manifestation.
Information taken from www.cancer.net.

| Prostate | |
|---|---|
| cancer stage | Clinical manifestation |
| Stage I | Slow growing. The tumor cannot be physically felt |
| | and involves one-half of a side of the prostate or |
| | less than one-half of a side of the prostate. |
| | Prostate-specific antigen (PSA) levels are low (i.e. |
| | PSA ≤ 10 ng/ml). The cancer cells do not show |
| | significant morphological abnormalities. |
| Stage II | Tumor localisation is restricted to the prostate. |
| | Low to medium (i.e. between 10 and 20 mg/ml) |
| | PSA levels. |
| Stage IIA | The tumor cannot be physically felt and involves |
| | half of 1 side of the prostate or less than one-half |
| | of a side of the prostate. Medium PSA levels and |
| | well differentiated cancer cells. This stage further |
| | includes larger tumors whereof localisation is |
| | restricted to the prostate. |
| Stage IIB | Tumor localisation restricted to the prostate and |
| | may be felt during digital rectal examination |
| | (DRE). Medium PSA levels. Moderately |
| | differentiated cancer cells. |
| Stage IIC | Tumor localisation restricted to the prostate, and |
| | may be felt during DRE. The PSA level is medium. |
| | Moderately or poorly differentiated cancer cells. |
| Stage III | High PSA levels. Growing tumor and/or high grade. |
| | Reasonable chance to grow and at risk of spreading. |
| Stage IIIA | Loss of restricted prostate localisation of malignant |
| | cells (i.e. tumors) and dissemination to nearby |
| | tissues, optionally including the seminal vesicles. |
| | High PSA levels (i.e. ≥ 20 ng/ml). |
| Stage IIIB | Further dissemination of malignant cells (i.e. |
| | tumors) to nearby structures, optionally including |
| | bladder and/or rectum. |
| Stage IIIC | Poorly differentiated cancer cells |
| Stage IV | Metastasis beyond the prostate. |
| Stage IVA | Metastasis to the regional lymph nodes. |
| Stage IVB | Metastasis to distant lymph nodes, other body parts |
| | and/or bones. |

In certain embodiments, the prostate cancer to be treated by a metal complex as described herein or a pharmaceutical composition as described herein is a prostate cancer having a Gleason score of from between 2 and 10, preferably a Gleason score of from between 4 and 10, more preferably a Gleason score of from between 6 and 10, most preferably a Gleason score of from between 7 and 10. The Gleason scoring system is known to a person skilled in the art (Munjal and Leslie, StatPearls, 2020).

Yet another aspect of the invention relates to a metal complex as described herein or a pharmaceutical composition as described herein for use in a method of in vivo diagnosis. Further envisaged are metal complexes as described herein or pharmaceutical compositions as described herein for use as a radiodiagnostic agent in a method of in vivo diagnosis. Therefore, also envisaged by the present invention are methods of diagnosis of PSMA-positive cancer types comprising administration of a detectable quantity of the metal complexes or pharmaceutical compositions as described herein to a subject and subsequent in vivo imaging of said metal complex. Also envisaged is the use of a metal complex as described herein for the manufacture of an in vivo diagnostic pharmaceutical composition. A further aspect of the invention relates to a metal complex as described herein or a pharmaceutical composition as described herein for use in a method of in vivo monitoring of PSMA expression and/or PSMA-expressing cells, more preferably PSMA-expressing cancer cells, most preferably PSMA-expressing prostate cancer cells. Further envisaged are metal complexes as described herein or pharmaceutical compositions as described herein for use as a radioimaging agent in a method of in vivo monitoring of PSMA expression and/or PSMA-expressing cells, preferably PSMA expressing cancer cells, most preferably PSMA-expressing prostate cancer cells. Therefore, also envisaged by the present invention are methods of in vivo monitoring PSMA expression and/or PSMA-expressing cells, preferably PSMA-positive cancer cells, most preferably PSMA positive prostate cancer cells comprising administration of a detectable quantity of a metal complex as described herein or a pharmaceutical compositions as described herein to a subject and subsequent in vivo imaging of said metal complex. In preferred embodiments, the use or method comprises conducting the step of administrating a detectable quantity of the metal complex or the pharmaceutical composition on at least two distinct time points. In further preferred embodiments, a first time point may be prior to the start of a given therapy that aims to reduce an aberrant amount and/or localisation of PSMA-expressing cells in a subject. Accordingly, a second, subsequent time point may be defined during or after the therapy. By means of illustration and not limitation, the imaging time points may be scheduled before and after a change in the type and/or dosage regiment of a therapy. By further means of illustration and not limitation, the imaging time points may be scheduled before and after a change in one or more changes in biomolecular parameters of a subject and/or before and after a change in clinical parameters of a subject. For example, the imaging time points may be scheduled at substantially regular intervals during or after a therapy, for example to monitor cancer regression, remission or relapse, preferably wherein the cancer is a PSMA-expressing cancer type, more preferably wherein the cancer type is prostate cancer. In certain embodiments, the in vivo monitoring is conducted in a context of preventive screening to detect formation of aberrant PSMA-expressing cells in a subject (i.e. a preventive and/or routine cancer screening procedure). In alternative embodiments, the in vivo monitoring is conducted in a context of therapy monitoring to assess therapy efficacy, optionally therapy efficacy of a radionuclide. In yet alternative embodiments, the in vivo monitoring is conducted in a context of periodical screening for recurrence (i.e. relapse) of a PSMA-expressing malignancy, such as but not limited to prostate cancer. Other appropriate embodiments of the imaging methods adapted for diagnosis and monitoring of any of the herein described indications will be apparent to the skilled person who is capable of defining further appropriate time points for imaging.

Further envisaged by the present invention is a metal complex as described herein or a pharmaceutical composition as described herein for use as a radiodiagnostic agent, preferably for use as a radiodiagnostic agent for in vivo imaging of cells expressing PSMA, preferably for use as a radiodiagnostic agent for in vivo imaging of cancer cells expressing PSMA, most preferably for use as a radiodiagnostic agent for in vivo imaging of prostate cancer cells expressing PSMA. Also envisaged is the use of a metal complex as described herein for the manufacture of a radiodiagnostic agent, optionally comprised in a radiodiagnostic composition.

“Diagnosed with”, “diagnosing”, and diagnosis are indicative for a process of recognising, deciding on, or concluding on a disease, condition, or (adverse side effect) in a subject on the basis of symptoms and signs and/or from results of various diagnostic procedures (such as, for example, from knowing the presence, absence and/or quantity of one or more biomarkers of or clinical symptoms characteristic for the diagnosed disease or condition). “Diagnosis of” the diseases, conditions, or (adverse) side effects as taught herein in a subject may particularly mean that the subject has such disease or condition. A subject may be diagnosed as not having such despite displaying one or more conventional symptoms or signs reminiscent of such. “Diagnosis of” the diseases or conditions as taught herein in a subject may particularly mean that the subject has respiratory infection disease. “Prognosticating” in the context of the invention is indicative for anticipation on the progression of a malignancy such as prostate cancer in a subject and the prospect (e.g. the probability, duration, and/or extent) of recovery, and/or the severity of experiencing or amelioration of said infection. The term “a good prognosis of” generally encompasses anticipation of a satisfactory partial or complete recovery from a diagnosed discase or pain condition, optionally within an acceptable time period. Alternatively, the term may encompass anticipation of not further worsening or aggravating of such, preferably within a given time period. The term “a poor prognosis of” the disease or condition typically encompass an anticipation of a substandard recovery and/or unsatisfactorily slow recovery, or no recovery at all, or further worsening of the malignancy and/or any clinical manifestation associated with said malignancy. “Radiodiagnosis” and “Radiodiagnostic agent” as used in the context of the present disclosure are terms that respectively indicate specific methods of diagnosis and diagnostic agents that allow a skilled (healthcare) practitioner to evaluate whether a subject is considered to have or has a specific medical condition by means of a clinical imaging method (i.e. by means of radiology) as defined further in the present disclosure. In certain embodiments, the diagnosis of aberrant PSMA expression, and therefore diagnosis of a PSMA-expressing cancer type is established by combining the images obtained after administration of the radiodiagnostic agent to a subject with any other means of diagnosis for a malignant neoplastic discase, such as prostate cancer. In further embodiments, the diagnosis of aberrant PSMA expression and therefore diagnosis of a PSMA-expressing cancer type such as prostate cancer is established by combining the images obtained after administration of the radiodiagnostic agent to a subject with a diagnosis method selected from the group of diagnosis methods consisting of: a digital rectal examination, a prostate-specific antigen (PSA) test, ultrasound imaging, magnetic resonance imaging, biopsy (e.g. transperincal biopsy or transrectal biopsy), or any combination thereof.

Related to the foregoing, “predicting” or “prediction” generally refer to a statement, declaration, indication or forecasting of a disease or condition in a subject not (yet) showing any, or a limited, clinical manifestation of said disease, condition, or (adverse) side effects. A prediction of a certain clinical disease manifestation, condition, or adverse effect in a subject may indicate a probability, chance, or risk that said subject will develop said clinical manifestation, condition, or (adverse) side effect, for example within a certain time period after diagnosis of the malignancy such as but not limited to prostate cancer. Said probability, chance or risk may be indicated as any suitable qualitative or quantitative expression, wherein non-limiting examples of a quantitative expression include absolute values, ranges or statistics. Alternatively, probabilities, chances, or risks may be indicated relative to a suitable control subject or group of control subject (i.e. a control subject population (such as, e.g., relative to a general, normal or healthy subject or subject population)). Therefore, any probability, chance or risk may be advantageously indicated as increased or decreased, upregulated or downregulated, as fold-increased or fold-decreased relative to a suitable control subject or subject population, or relative to a baseline value which may be derived from either a control subject (population), textbook reference values. It is evident that when a population of subjects is used to define the baseline value, said baseline value will be a centre size of one or more values (parameters) of a population, such as the mean or median of said value. A skilled person further appreciates that monitoring of a malignancy such as but not limited to prostate cancer may allow to predict the progression, aggravation, alleviation or recurrence of the clinical image or severity of said malignancy. Furthermore, monitoring may be applied in the course of a medical treatment of a subject. Such monitoring may be comprised, e.g., in decision making whether a patient may be discharged from a controlled clinical or health practice environment, needs a change in treatment or therapy, or requires (extended) hospitalisation.

A further aspect of the invention is directed to a metal complex as described herein or a pharmaceutical composition as described herein for use as a theranostic agent. Also envisaged are methods of simultaneous diagnosis and treatment, comprising administering a metal complex as described herein or a pharmaceutical composition as described herein in a detectable and pharmaceutically active amount to a subject. Further intended is the use of a metal complex as described herein for the manufacture of a theranostic composition. The term “theranostic agent” is known to a skilled person (described in detail e.g. in Langbein et al., J Nucl Med. 2019) and refers in the context of the present invention to a suitability of a single metal complex as described herein which preferentially or selectively binds to PSMA that is suitable for use as both a diagnostic agent, a therapeutic agent, and optionally a means for monitoring disease progression and/or therapy efficacy. Several suitable atoms for use in theranostics have been described and include without limitation lutetium-177 (¹⁷⁷Lu), actinium-255 (²²⁵ Ac), iodine-123 (¹²³I), iodine-131 (¹³¹I), yttrium-86, yttrium-90, terbium-152 (¹⁵²Tb), terbium-155 (¹⁵⁵Tb), terbium 149 (¹⁴⁹Tb), and terbium-161 (¹⁶¹Tb). Criteria for suitability of an atom as part of a theranostic agent have been described in the art (Yordanova et al., Onco Targets Ther. 2017).

Yet a further aspect of the invention is directed to a metal complex as described herein for use in radioguided surgery. Therefore also envisaged are methods of radioguided surgery comprising administration of a detectable quantity of the metal complex and a subsequent step of invasive surgery to remove PSMA-expressing tumor tissue that is radiolabeled by said probe. Hence, the use of a metal complex as described herein for the manufacture of a pharmaceutical labelling composition for use in radiosurgery is accordingly envisaged. “Radioguided surgery” is a medical procedure known to a skilled person and is has been described at numerous occasions (e.g. in Povoski et al., World J Surg Oncol, 2009). Briefly, radioguided surgery relies on radiolabeling a certain target tissue or target cell type, in the context of the present invention typically PSMA-expressing cells and/or PSMA-expressing tumor cells, whereafter said radiolabeled tissues or cells are surgically removed. During the surgical procedure, a probe is utilised to “scan” the surgical wound area for radiolabeled tissues and cells, which indicates to the surgeon(s) which tissue was radiolabeled, and hence in the context of PSMA-expressing cancer tissue or cells need to be surgically removed.

The term “imaging” as used ubiquitously throughout the present disclosure is to be interpreted in its broadest context and hence encompasses any medical imaging technique or process for creating visual representations of the interior of a body and/or visual representation of the function of organs or tissues of a subject. Non-limiting examples of imaging methodologies and techniques as envisaged by the present disclose include X-ray radiography, X-ray computed tomography (CT), magnetic resonance imaging (MRI), positron emission tomography (PET), PET-CT, and single-photon emission computed tomography (SPECT). In preferred embodiments, the imaging modality may be PET, PET-CT, or SPECT since these imaging methods are particularly suited for visualising a detectable signal of the metal complexes described herein. In more preferred embodiments, the emitted signal by a detectable quantity of a metal complex described herein is detected by positron emission tomography (PET) and a PET image is generated. In alternative preferred embodiments, the emitted signal by a detectable quantity of a metal complex described herein is detected by single photon emission computed tomography (SPECT) and a SPECT image is generated. In certain embodiments, the imaging methods described herein may further comprise superimposing a PET or SPECT image with a computed tomography (CT) image or a magnetic resonance image (MRI).

In certain embodiments, the imaging methods described herein may be used to monitor, follow-up or track the progression of a malignancy such as but not limited to prostate cancer over time by generating images that lend themselves to a side-by-side comparison (e.g., images generated with the same quantity of the antibody per kg subject weight and the same route and manner of administration: using substantially the same settings on the imaging system; etc.) at two or more sequential time points, optionally where the patient has received or may be receiving a treatment aimed at slowing and/or inhibiting disease progression.

In certain embodiments, two or more distinct metal complexes may be detected in the imaging methods described herein. A simultaneous or consecutive detection of two or more metal complexes enables detection and optionally visualisation of multiple entities such as but not limited to distinct molecular markers, distinct cell types, and/or distinct tissues. Hence, in certain embodiments, the imaging methods described herein comprise detecting at least two metal complexes, of which at least one metal complex binds preferentially or selectively to PSMA. In alternative embodiments, the imaging methods comprise detection of at least one metal complex which binds preferentially or selectively to PSMA and one further signal emitting molecule which does not bind preferentially or selectively to PSMA.

In preferred embodiments wherein the metal complex or pharmaceutical composition is used in the treatment, prevention, and/or diagnosis of cancer or tumor, the cancer or tumor is a PSMA-expressing cancer or tumor. In further preferred embodiments, the cancer specified herein is selected from the group of cancers selected from the group consisting of: renal cancer, bladder cancer, lung cancer, and cancers of the oral cavity, and prostate cancer. In further preferred embodiments, the cancer specified herein is selected from the group of cancers selected from the group consisting of: conventional renal cell cancer, transitional cell of the bladder cancer, non-small-cell lung cancer, testicular-embryonal cancer, neuroendocrine cancer, colon cancer, prostate cancer, and breast cancer. In highly preferred embodiments, the cancer is prostate cancer.

A skilled person is aware that certain individuals may experience yet improved benefits from medical treatment by the metal complex by further optimisation of the dose of said component by considering a wide range of parameters including but by no means limited to the disease stage of the subject, gender, agc, body weight, other medical indications, nutrition, mode of administration, metabolic state, interference or influence by or efficacy of other pharmaceutically active ingredients, etc. Furthermore each individual may have a certain intrinsic degree of responsiveness to the metal complex that is used.

It is envisaged by the present disclosure that a metal complex as described herein or a pharmaceutical composition as described herein can be combined with one or more anti-cancer treatment methods or anti-cancer therapies, including but not limited to surgery, radiotherapy, chemotherapy, biological therapy, or any combinations thereof. Therefore, in certain embodiments the metal complex as described herein, optionally comprised in a pharmaceutical composition, is used and/or administered as the sole active pharmaceutical agent. In equally envisaged embodiments, the metal complex as described herein, optionally comprised in a pharmaceutical composition, is used and/or administered in conjunction with at least one additional active pharmaceutical agent on condition that the combined use of the metal complex and the additional active pharmaceutical agent does not invoke any adverse effects on the subject. The term “chemotherapy” should be interpreted broadly and hence encompasses any treatment relying on the use of a chemical substance or composition. Therefore, in certain embodiments, a chemotherapeutic agent that is combined with the metal complex as described herein or the pharmaceutical composition as described herein is selected from the group consisting of: alkylating agents, cytotoxic compounds, anti-metabolites, plant alkaloids, terpenoids, topoisomerase inhibitors, or any combination thereof. The term “biological therapy” should be interpreted equally broadly and encompasses treatments relying on the use of biological substances or compositions comprising one or more biological substance, such as biomolecules, or biological agents. By means of illustration, such biological substances include viral cells, and illustrative biomolecules include peptides, polypeptides, proteins, nucleic acids, small molecules (e.g. metabolites or natural products), or any combination thereof. In certain embodiments wherein a metal complex or a pharmaceutical compositions comprising a metal complex as described herein is used in conjunction with a biomolecule, the biomolecule is selected from the group consisting of: interleukins, cytokines, anti-cytokines, tumor necrosis factor (TNF), cytokine receptors, vaccines, interferons, enzymes, therapeutic antibodies, antibody fragments, antibody-like protein scaffolds, or any combination thereof. In further embodiments, the biomolecule is selected from the group consisting of: aldesleukine, alemtuzumab, atezolizumab, bevacizumab, blinatumomab, brentuximab vedotine, catumaxomab, cetuximab, daratumumab, denilcukin diftitox, denosumab, dinutuximab, clotuzumab, gemtuzumab ozogamicin, 90Y-ibritumomab tiuxetan, idarucizumab, interferon α, ipilimumab, necitumumab, nivolumab, obinutuzumab, ofatumumab, olaratumab, panitumumab, pembrolizumab, ramucirumab, rituximab, tasonermin, 131I-tositumomab, trastuzumab, Ado-trastuzumab emtansine, and any combination thereof.

Further examples of anti-cancer therapy strategies include hormone therapy, immunotherapy, and stem cell therapy, which are commonly considered as falling within the umbrella term “biological therapies” and are each suitable for use in combination with a metal complex as described herein or a pharmaceutical composition described herein. In certain embodiments a metal complex described herein or pharmaceutical composition as described herein may therefore be used in conjunction with a hormone therapy. Illustrative examples of such substances include without limitation tamoxifen, aromatase inhibitors, luteinizing hormone blockers, anti-androgens, gonadotrophin releasing hormone blockers, and any combination thereof. In alternative certain embodiments a metal complex as described herein or a pharmaceutical composition as described herein may therefore be used in conjunction with immunotherapy, said therapy broadly indicating any treatment that is capable of modulating the immune system of a subject. Particular, the term “immunotherapy” comprises any treatment that modulates an immune response, such as a humoral immune response, a cell-mediated immune response, or any combination thereof. “Immunotherapy” additionally comprises cell-based immunotherapies in which immune cells are transferred into the patient, for example T cells and/or dendritic cells. The term also comprises an administration of substances or compositions, such as chemical compounds and/or biomolecules (e.g., antibodies, antigens, interleukins, cytokines, or combinations thereof), that modulate a subject's immune system. Illustrative examples include the use of monoclonal antibodies (e.g. Fc-engineered monoclonal antibodies against proteins expressed by tumor cells), immune checkpoint inhibitors, prophylactic or therapeutic cancer vaccines, adoptive cell therapy, and combinations thereof. A further examples of a therapy which is suitable for combination with the metal complexes described herein or the pharmaceutical compositions described herein is adoptive cell therapy. Adoptive cell therapy generally refers to the transfer of cells such as immune-derived cells (e.g. cytotoxic T cells), back into the same patient or into a new recipient host with the goal of transferring the immunologic functionality and characteristics into the new host. Alternatively, chimeric antigen receptors may be used in order to generate immune responsive cells (such as T cells; CAR-T) specific for selected targets such as malignant cells. Methods to genetically modify T cells have been described in the art (e.g. in Li et al. Signal Transduct Target Ther, 2019). Hence, in certain embodiments a metal complex as described herein or a pharmaceutical composition as described herein may therefore be used in conjunction with adoptive cell therapy or CAR-T cell therapy. A final illustrative treatment strategy which may be employed in combination with one or more presently described metal complexes or presently described pharmaceutical compositions is stem cell therapy. In cancer stem cell therapy, bone marrow stem cells are destroyed by e.g. radiation therapy or chemotherapy, prior to transplantation of stem cells (autologous, syngencic, or allogeneic) into the subject.

A skilled person is knowledgeable of administration routes, doses, and treatment regimens of anti-cancer agents known in the art since these have been described in detail at numerous instances (e.g. in Schellens et al., Oxford University Press “Cancer Clinical Pharmacology”, 2005). It is further evident that any of the above combination therapies may be administered prior to, simultaneously with, or after administration of a metal complex described herein or pharmaceutical composition comprised herein.

A further aspect of the invention relates to a radiolabeling kit comprising a compound (labelling precursor) of formula (I), and a suitable buffering system. In preferred embodiments, the radiolabelling kit is designed for coordination of technetium-99m or rhenium-188. In preferred embodiments, the compound of formula (I) and/or the suitable buffering system comprised in the kit are contained in glass vials, optionally provided with a deformable stopper as closure means, preferably a rubber stopper as closure means. In further preferred embodiments, the compound of formula (I) and/or the suitable buffering system comprised in the kit are contained in siliconized vials such as borosilicate glass vials, more preferably type I neutral borosilicate glass vials. In further preferred embodiments, the kit comprises water for injection which may be included in the kit of parts as suitable buffering system or as a further component of the kit. A skilled person is aware of the regulatory standards set for water for injection and is therefore knowledgeable of the criteria that need to be adhered to according to USP 30 and/or EP addendum 2001. In certain embodiments, the water for injection is USP 30 water for injection. Alternatively, the water for injection is EP addendum 2001 water for injection. In certain embodiments, at least one of the components are freeze dried (i.e. lyophilised). In alternative embodiments, the kit of parts may comprise a formate, phosphate, HEPES and/or acetate buffer as suitable buffering system. Hence in certain embodiments, the suitable buffering system is selected or comprises a buffering agent selected from the group consisting of: water for injection, monobasic sodium phosphate, dibasic sodium phosphate, sodium acetate, acetic acid, or any combination thereof. It is evident that any of the components of the kit may further contain additional excipients to improve long term storage of said component(s), improve the range of storage conditions which are possible, stability of the component(s) before, during, or after admixing or constitution of the final pharmaceutical product, or any combination thereof.

In some embodiments, the kit comprises the following components:
the labeling precursor (or compound) as defined herein in any one of the aspects or embodiments;
a reducing agent, enabling the reduction of the pertechnetate/perrhenate to Tc(V)O/Re(V)O such as ascorbic acid, sodium borohydride, sodium dithionite, phosphines such as TCEP, and stannous chloride (Tin(II)chloride), preferably stannous chloride most preferably stannous chloride (tin(II)chloride),
an antioxidant, enabling the protection of the product against radiolytix oxidation such as ascorbic acid, sodium borohydride, sodium dithionite, and stannous chloride,
optionally auxillary agents or ligands enabling the protection against reoxidation of Tc(V)O/Re(V)O as competing reaction to coordination, such as for tartrate/citrate/glucoheptonate,
optionally a stabilizer enabling the storage of the kit known in the art,
optionally further excipients such as lyophilization agents, matrix reagents or solubilizers known in the art.

Additional substances such as sequesters of metal impurities derived from the radionuclide generator, can be present as well. Non-limiting examples of such sequestering agents can be mono-, di-, or oligosaccharides as disclosed in e.g. WO2016030103A1 and WO2016030104A1 or polysaccharides and other polynucleate sequestering agents as disclosed in e.g. WO2013024013A2. Such sequestering agents will typically compete with the chelator part of the labelling precursor for the impurities derived from the radionuclide generator thereby avoiding the need for cumbersome purification after radiolabelling.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples.

### EXAMPLES

### Example 1: Synthesis of the Labeling Precursors

The synthesis of the pharmacophore was accomplished by a well-known procedure such as reported in Eder et al., 2014 (Prostate 2014 May;74(6):659-68): The isocyanate of the glutamyl moiety was generated in situ by adding a mixture of 3 mmol of bis(tert-butyl) L-glutamate hydrochloride and 1.5 mL of N-ethyldiisopropylamine (DIPEA) in 200 mL of dry CH₂Cl₂ to a solution of 1 mmol triphosgene in 10 mL of dry CH₂Cl₂ at 0° C. over 4 h. After agitation of the reaction mixture for 1 h at 25° C., 0.5 mmol of the resin-immobilized (2-chloro-tritylresin) E-allyloxycarbonyl protected lysine in 4 mL DCM was added and reacted for 16 h with gentle agitation. Subsequently the resin was filtered off and dried.

The syntheses of the labeling precursors were conducted with aliquots of the resin carrying approx. 50 μmol pharmacophore. For deprotection, the resin was swelled in CH₂Cl₂ (Dichloromethane, DCM) and reacted with a mixture of 10 mg (PPh₃)₄Pd⁰ and 60 mg dimethylaminoborane in 3 ml DCM for 15-30 minutes. Subsequently the resin was washed with DCM, 5% aminoethanol in DCM (5 min shaking), Methanol, Dimethylformamide (DMF) (3-5 times each).

The linkers were build-up by means of standard solid phase peptide synthesis (SPPS) using fluorenylmethoxycarbonyl (Fmoc) as protective group. Protective groups for the side-chains were tert-butyl for carboxylic acids and tert-butoxycarbonyl (Boc) for amino-groups. Each coupling was conducted with 3 equivalents of the respective Fmoc protected aminoacid, 2.96 equivalents of HATU and 8-12 equivalents of diisopropylamine (DIPEA) for 30 minutes at room temperature under agitation in DMF. Removal of the fmoc group was conducted by reaction with 20% piperidine in DMF for 5 minutes at roomt temperature (3 times each). Between the individual steps, the resin was washed with DMF (5 times each).

The chelator usually consisting of 2-3 aminoacids and a termial mercaptoacetyl group was build using the same procedure as described for the linker. The coupling of the mercaptoacetylgroup was conducted using acetyl protected 2-mercaptoacetic acid (same procedure as for the linker/chelator). For removal of the terminal acetyl-protective group, the solvent was changed to acetonitrile (MeCN) and the deprotection was conducted with 35 μl hydrazinchydrate in 2 ml MeCN for 20-20 minutes at room temperature under agitation. Then the resin was washed with MeCN, DMF and DCM (5 times each). Finally, the compounds were cleaved from the resin using TFA containing 2.5% water and 2.5% triisopropylsilane (TIS) (2-3 ml) for 30-45 min at room temperature. The cleavage cocktail was filtered, diluted with 20 ml DCM and the solvents removed under reduced pressure. The crude product was purified by preparative HPLC. Identity of the compounds was confirmed by HPLC-MS.

### Example 2: Syntheses of the ^{nat}Re-Coordinated Reference Compounds

Approx. 0.1-0.2 μmol of the respective precursor (GCK-XX) were reacted with 2 eq. of Trichloro-oxo-bis-(triphenylphosphine)-rhenium(V) in 200 μl water/methanol (1:1) at 96° C. for 90 minutes. The pH of the solution was adjusted to 8.0-8.5 using NaOH. The product was extracted by solid phase extraction (Waters SepPak plus light tC18 preconditioned with 5 ml EtOH and 10 mL water), washed with approx. 2 mL saline and cluted in 1 ml 70% ethanol. The product was analyzed using HPLC/MS and used to demonstrate co-elution with the respective labeled compounds.

### Example 3: Radiosyntheses of the ^{99m}Tc-Coordinated Compounds for In Vitro Application

Phosphate buffer was prepared from 890 mg Na₂PO₄.2H₂O in 9.5 mL water for injection and 0.5 mL 2 m NaOH. After dissolution of the salts, the buffer was sterile filtered, the pH was determined using pH stripes (pH=11.5-12.0). The labeling mixture consisted of 1 μl precursor solution (1 mg/ml in MeCH/H₂O 20:50; approx. 1 nmol), 20 μl phosphate buffer, 10 μl tris-carboxyphenylphosphin (TCEP; 28.7 mg/mL in phosphate buffer; 0.1 molar solution) and 4-10 μL pertechnetate in saline (0.9% NaCl; generator cluate) containing an activity of approx. 7.5 MBq. The mixture was filled to 100 μl with saline (0.9% NaCl; 59-65 μL). The pH of the reaction mixture was 8.0-8.5 (tendency towards 8.5). The mixture was heated at 98° C. for 10 minutes. After cooling to room temperature, the mixture was diluted to 1 mL by addition of saline (0.9% NaCl, ligand concentration 1 μm). An aliquot was analyzed by HPLC to determine the radiochemical yield (RCY) (method B, see example 9). Aliquots of the product mixture containing the ^{99m}Tc-labeled ligand were further diluted to a concentration of approx. 100 nM (precursor) in PBS with and without a 10000 fold excess of 2-(Phosphonomethyl)-pentandioic acid, 2-Phosphonomethyl pentanedioic acid (2-PMPA) as competitor.

RCY and RCP were determined via analytical HPLC. Since the RCY was usually >95% the product could be used without further purification (RCY=RCP in this case).

### Example 4: Cellular Uptake Experiments/Competitive Binding

Cellular uptake experiments were conducted in analogy to previously described procedures (Lindner et al., Doi.: 10.2967/jnumed.118.210443). Briefly, LNCaP cells were seeded in 6-Well plates in RPMI medium containing 20% FBS (two day of incubation prior to the experiment) and grown to a confluency of 70-80%. For the uptake experiment, the medium was removed and the cells were incubated with 1 mL of a 1:99 dilution of the 100 nM product mixture dilutions (with and without competitor) described in Example 3. After an incubation period of 1 h the medium containing the ^{99m}Tc-ligand was removed, the cells were washed two times with 1 mL PBS and lysed using lysis buffer (two times 700 μL each; 3.0 M NaOH containing 0.2% SDS). The cellular uptake was determined from the activity in the lysed fraction. The unspecific uptake was determined from the cellular uptake of the cells incubated with the ligand in presence of the competitor. For the determination of the specific uptake, the unspecific uptake was subtracted the cellular uptake. Each experiment was conducted as triplicate. The results are depicted in Table 2.

For determination of Ki, competitive binding experiments were conducted. Four six-well plates were seeded as described above. The cells were incubated with 1 mL of a 1:99 dilutions of the 100 nM product mixture described in Example 3 in medium containing different concentrations of competitor (2-PMPA, 10E-4/-5/-6/-7/-8/-9 mol/L). After an incubation time of 1 h the medium was removed, the cells washed with two times with 1 mL PBS and lysed using lysis buffer (two times 700 μL each; 3.0 M NaOH containing 0.2% SDS). The cellular uptake was determined from the activity in the lysed fraction. From this data-set the 50% inhibitory concentration (IC50) was determined and the Ki calculated using the cheng-prusoff equation.

TABLE 2
Cellular uptake of synthesized candidate molecules.

| | | Unspecific | Specific | |
|---|---|---|---|---|
| | Uptake | uptake | uptake | |
| | [% AD/ | [% AD/ | [% AD/ | Ki |
| Substance | 10⁶ cells] | 10⁶ cells] | 10⁶ cells] | [nm] |
| [^{99m}Tc]TLX-598 | 20.3 ± 0.3 | 1.64 ± 0.07 | 18.7 ± 0.3 | (38) |
| (reference compound) | | | | |
| [^{99m}Tc]Tc-GCK01 | 19.6 ± 4.8 | 1.2 ± 0.6 | 18.4 ± 4.2 | (26) |
| [^{99m}Tc]Tc-GCK02 | 16.5 ± 0.9 | 3.3 ± 0.2 | 13.2 ± 1.0 | |
| [^{99m}Tc]Tc-GCK03 | 25.3 ± 3.7 | 6.1 ± 0.4 | 19.2 ± 3.3 | |
| [^{99m}Tc]Tc-GCK05 | 22.8 ± 1.1 | 1.2 ± 0.1 | 21.5 ± 1.2 | |
| [^{99m}Tc]Tc-GCK06 | 25.3 ± 1.4 | 7.6 ± 0.9 | 17.7 ± 2.3 | |
| [^{99m}Tc]Tc-GCK07 | 8.2 ± 1.6 | 1.3 ± 0.1 | 6.9 ± 1.6 | |
| [^{99m}Tc]Tc-GCK09 | 30.0 ± 10.7 | 3.8 ± 1.5 | 26.2 ± 9.3 | |
| [^{99m}Tc]Tc-GCK011 | 57.8 ± 12.4 | 11.9 ± 3.9 | 45.9 ± 8.5 | |

### Example 5: ^{99m}Tc-Labeling for In Vivo Application

Phosphate buffer was prepared from 890 mg Na₂PO₄.2 H₂O in 9.5 mL water for injection and 0.5 mL 2 m NaOH. After dissolution of the salts, the buffer was sterile filtered, the pH was determined using pH stripes (pH=11.5-12.0). The labeling mixture consisted of 20 μl precursor solution (1 mg/ml in MeCH/H₂O 20:50), 200 μl phosphate buffer, 100 μl tris-carboxyphenylphosphin (TCEP; 28.7 mg/mL in phosphate buffer; 0.1 molar solution) and 500-800 μL pertechnetate in saline (0.9% NaCl; generator eluate). The pH of the reaction mixture was 8.0-8.5 (tendency towards 8.5). The mixture was heated at 98° C. for 10 minutes. After cooling to room temperature an aliquot was analyzed by HPLC to determine the radiochemical yield (method B, see example 9). The remaining solution was diluted with approx. 0.5-1.0 ml saline (0.9% NaCl), passed through and SepPak plus light tC18 cartridge (Waters corp., Eschborn, Germany) preconditioned with 5 mL ethanol and 10 ml water for injection), washed with 2 mL saline (0.9% NaCl) and eluted in 1 mL 70% Ethanol (prepared from Ethanol Ph. Eur. (VWR) and water ad injectabilia (BBraun)). An aliquot of the eluate was analyzed by HPLC to determine the amount of remaining TCEP (not detectable in all cases; spike was detectable). The remaining solution was diluted with 9 mL PBS (prepared from 9 mL 0.9% NaCl and 1 mL sodiumphosphate concentrate: BBraun ad injectabilia, both) and filtered via a 0.22 μm sterile filter in a 15 mL glass vial.

### Example 6: ¹⁸⁸Re-Labeling for In Vivo Application

¹⁸⁸Re was eluted from a ¹⁸⁸W/¹⁸⁸Re radionuclide generator (OnkoBeta, OnkoBeta GmbH, Garching, Munich) using 10 mL 0.9 & NaCl (BBraun). The generator eluate was postprocessed according to the procedures described by Guhlke et al. (S. Guhlke et al. JNM 2000, 47. 1271-1278). Briefly, potential tungsten breakthrough was retained on an SepPak Alumina(N) cartridge. The eluate was dechlorinated using a Dionex OnGuard II Ag cartridge and the perrhenate was concentrated using a SepPak QMA cartridge, preconditioned with 5 mL 1 m K₂CO₃, followed by 10 mL deionized water. The perrhenate was eluted from the cartridge using 1 mL of 0.9% NaCl (BBraun). The recovery during this process was usually >80%.

A typical ¹⁸⁸Re-labeling mixture consisted of 30 μL citrate solution (100 mg/mL), 10 μL GCK-XX precursor solution (1 mg/mL in MeCN/H₂O 50:50 v/v), 10 μl 30% ascorbic acid solution (in water), 200 μL perrhenate in 0.9% NaCl (postprocessed as described above) and 12 μL SnCl₂ (50 mg/mL in 1 m HCl). The pH of the mixture was usually 2.0-3.5. The mixture was heated for 60 min at 96° C. After cooling to ambient temperature, the mixture was diluted with 1 mL 0.9% NaCl and passed through a SepPak plus light tC18 cartridge. The cartridge was washed with 2-3 mL 0.9% NaCl and the product cluted with 1 mL 70% EtOH. The solution containing the product was usually diluted et least 1:9 into PBS (prepared from 9 mL 0.9% NaCl and 1 mL sodiumphosphate concentrate; BBraun ad injectabilia, both) containing 1-3 vol % 30% ascorbic acid solution.

The (radiochemical) yield was determined by division of the isolated product activity by the starting activity. Due to the long half-life, decay correction was omitted (Approx. 4% decay per h). The radiochemical purity was determined by radio HPLC for the isolated product (after cartridge separation and formulation).

### Example 7: In-Vivo Planar Imaging

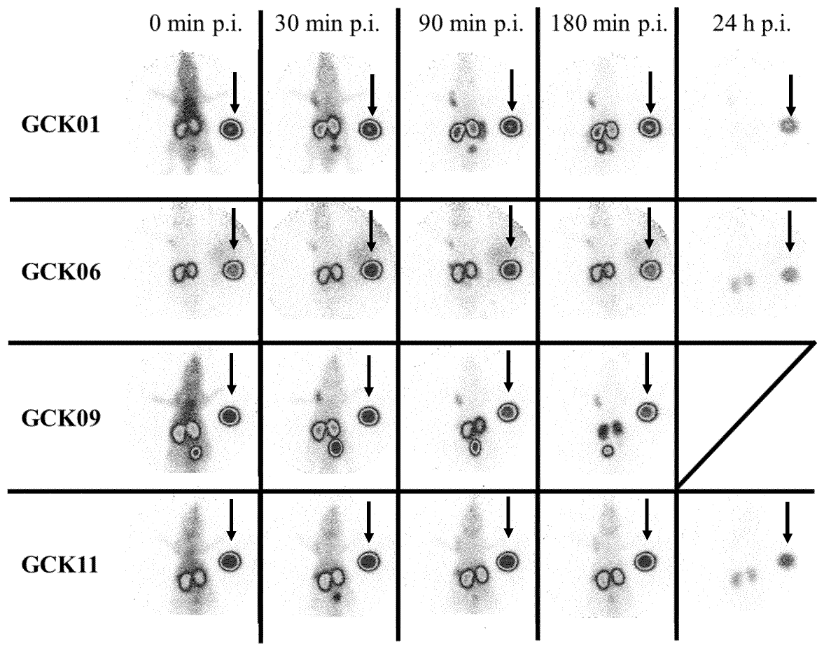

For the in-vivo planar imaging, 100 μl of a formulation containing 5-10 MBq ^{99m}Tc-labelled compound (approx. 0.1 μg precursor, 1 μg precursor/mL; formulation in PBS as described in example 5) were injected into the tail vein of a LnCAP Tumor bearing mouse. The animals were anesthetized with Sevorane (Abbvie, Wiesbaden, Germany), placed on the Gamma IMAGER—S/C (Paris, France)in prone position to perform planar imaging (using Gamma Acquisition und GammaVision+ software). An activity standard (approx. 1 MBq of the respective tracer) was prepared in a closed HPLC-sample flask and placed next to the animal during all timepoints of the measurement. The results of the planar imaging at different time points with [^{99m}Tc]Tc-GCK01, 6, 9 and 11 are shown in FIG. 1.

From this we can deduct that all compounds have good tumor radiolabeling capabilities and show rapid clearance.

### Example 8: Ex-Vivo Organ Distribution

For ex-vivo biodistribution, LnCAP tumor bearing mice were injected with 100 μl of a formulation containing approx. 1 MBq of the respective ¹⁸⁸Re-labelled compound GCK01 (approx. 0.1 μg precursor, 1 μg precursor/mL; formulation in PBS as described in example 6), each. The animals were sacrificed at 1 h p.i, and 3 h p.i., respectively. Organs of interest were dissected, blotted dry, weighted and the radioactivity was determined on a gamma counter (Packard Cobra II, GMI, Minnesota, USA) and calculated as % ID/g. The results are provided in Table 3.

TABLE 3
Ex-vivo biodistribution of approx. 1
MBq of the respective [¹⁸⁸Re-]-GCK01.

| | % ID/g | % ID/g |
|---|---|---|
| Organ | 1 h p.i. | 3 h p.i. |
| Urin | 36 ± 24 | 71 ± 18 |
| Blood | 2.1 ± 0.7 | 0.74 ± 0.08 |
| Heart | 0.9 ± 0.3 | 0.46 ± 0.03 |
| Lung | 2.0 ± 0.6 | 1.01 ± 0.08 |
| Spleen | 11 ± 3 | 3.9 ± 1.7 |
| Liver | 0.68 ± 0.05 | 0.28 ± 0.01 |
| Kidneys | 70 ± 23 | 91 ± 7 |
| Muscle | 0.3 ± 0.1 | 0.18 ± 0.06 |
| Intestines | 0.36 ± 0.06 | 0.24 ± 0.04 |
| brain | 0.04 ± 0.02 | 0.030 ± 0.004 |
| Stomach | 1.0 ± 0.5 | 0.8 ± 0.5 |
| Tumor | 5.1 ± 1.4 | 10.7 ± 1.9 |

### Example 9: GCK01

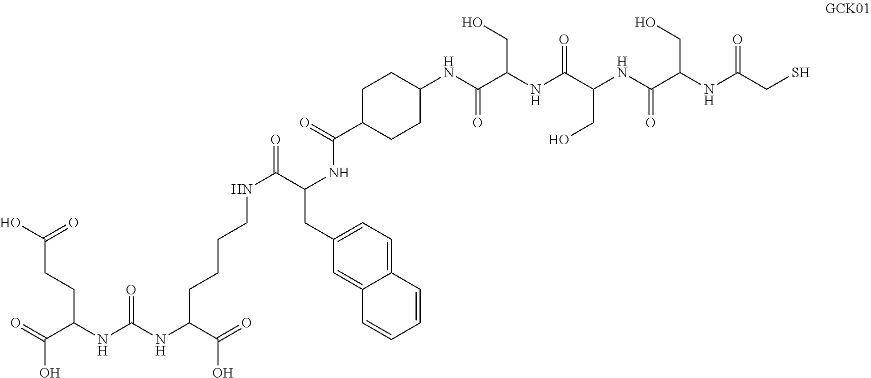 [SMILES: O=C(O)CCC(NC(=O)NC(CCCCNC(=O)C(Cc1ccc2ccccc2c1)NC(=O)C1CCC(NC(=O)C(CO)NC(=O)C(CO)NC(=O)C(CO)NC(=O)CS)CC1)C(=O)O)C(=O)O]

All syntheses were conducted as described under the examples 1-5.

Precursor (sequence): Glu-(urea)-Lys-2Nal*-tACHC-ser-ser-ser-MA (*=bound to the ε-amino group of the neighboring lysine; 2Nal=L-2-Naphtylalanine; tACHC=trans-4-aminocyclohexane carboxylic acid; MA=2-Mercaptoacetic acid)

Chemical formula: C₄₃H₆₀N₈O₁₆S (Mw: 977.04 g/mol)

MS (HPLC-ESI-MS): m/z ([M+H⁺]⁺)=977.26 (found); 977.37 (calc.); tᵣ=11.66 min (gradient A: 0% A (0 min) 100% A (20 min) linear gradient, 0.2 ml/min; A+B=100%; solvent A: MeCN+0.1% trifluoroacetic acid, solvent B: water+0.1% trifluoroacetic acid; Column: Hypersil Gold aQ 200×2.1 mm, 1.9 μm particle size)

Purity (HPLC): >90%, tᵣ=2.24 min (gradient B: 0% A (0 min) 100% A (5 min) linear gradient, 2 ml/min; A+B=100%; solvent A: MeCN+0.1% trifluoroacetic acid, solvent B: water+0.1% trifluoroacetic acid; Column: Chromolith Performance C18e 100×3 mm)

### Cold Reference Standard (^{nat}Re Coordinated)

Chemical formula: C₄₃H₅₇N₈O₁₇ReS (Mw: 1176.23 g/mol)

MS (HPLC-ESI-MS): m/z=1177.29 (found); 1177.32 (calc.); tᵣ=11.64 min (gradient: A—see above)

Purity (HPLC): >90%, tᵣ=2.30 min (gradient: B—see above)

### [¹⁸⁸Re]Re-GCK01

RCY/RCP: 75% />95%

HPLC (gradient B): tᵣ=2.12/2.17 min

### [^{99m}Tc]Tc-GCK01

RCY/RCP:>95%

HPLC (gradient B): tᵣ=2.33 min

### Example 10: GCK02

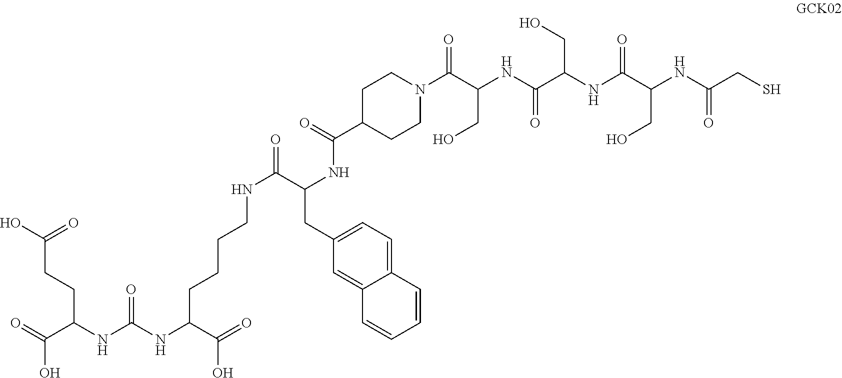 [SMILES: O=C(O)CCC(NC(=O)NC(CCCCNC(=O)C(Cc1ccc2ccccc2c1)NC(=O)C1CCN(C(=O)C(CO)NC(=O)C(CO)NC(=O)C(CO)NC(=O)CS)CC1)C(=O)O)C(=O)O]

All syntheses were conducted as described under the examples 1-5.

Precursor (sequence): Glu-(urea)-Lys-2Nal*- Inp-ser-ser-ser-MA (*=bound to the s-amino group of the neighboring lysine; 2Nal=L-2-Naphtylalanine; Inp=isonipecotic acid; MA=2-Mercaptoacetic acid)

Chemical formula: C₄₂H₅₈N₈O₁₆S (Mw: 963.02 g/mol)

MS (HPLC-ESI-MS): m/z ([M+H⁺]⁺)=963.38 (found); 963.35 (calc.); tᵣ=11.58 min (gradient A, see example 9)

Purity (HPLC): >95%, tᵣ=2.455 min (gradient B, see example 9)

### [¹⁸⁸Re]Re-GCK02

RCY/RCP: n.d./>90%

HPLC (gradient B, see Example 9): tᵣ=2.216 min

### [^{99m}Tc]Tc-GCK02

RCY/RCP: >90%

HPLC (gradient B, see Example 9): tᵣ=2.212 min

### Example 11: GCK03

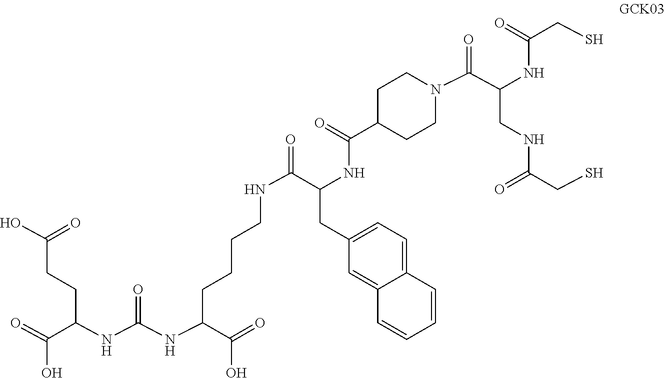 [SMILES: O=C(O)CCC(NC(=O)NC(CCCCNC(=O)C(Cc1ccc2ccccc2c1)NC(=O)C1CCN(C(=O)C(CNC(=O)CS)NC(=O)CS)CC1)C(=O)O)C(=O)O]

All syntheses were conducted as described under the examples 1-5.

Precursor (sequence): Glu-(urea)-Lys-2Nal*-(Inp)-Dap(MA)-MA (*=bound to the ε-amino group of the neighboring lysine; 2Nal=L-2-Naphtylalanine; Inp=isonipecotic acid; MA=2-Mercaptoacetic acid)

Chemical formula: C₃₈H₅₁N₇O₁₂S₂ (Mw: 861.91 g/mol); Disulfide C₃₈H₄₉N₇O₁₂S₂ (Mw: 859.96 g/mol)

MS (HPLC-ESI-MS): m/z ([M+H⁺]⁺)=860.270 (found); 860.296 (calc., disulfide); tᵣ=12.5 min (gradient A, see example 9)

Purity (HPLC): >90%, tᵣ=2.222 min (gradient B, see example 9)

### [¹⁸⁸Re]Re-GCK03

RCY/RCP: n.d./>90%

HPLC (gradient B, see Example 9): tᵣ=2.523 min

### [^{99m}Tc]Tc-GCK03

RCY/RCP: >95%

HPLC (gradient B, see Example 9): tᵣ=2.52 min

### Example 12: GCK05

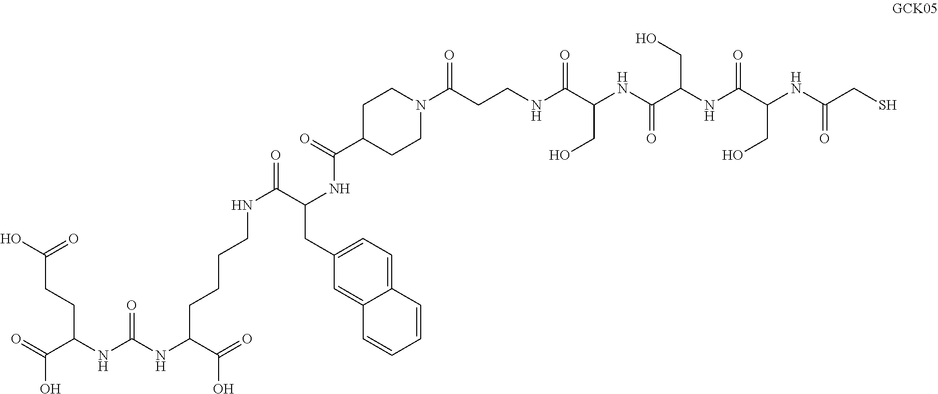 [SMILES: O=C(O)CCC(NC(=O)NC(CCCCNC(=O)C(Cc1ccc2ccccc2c1)NC(=O)C1CCN(C(=O)CCNC(=O)C(CO)NC(=O)C(CO)NC(=O)C(CO)NC(=O)CS)CC1)C(=O)O)C(=O)O]

All syntheses were conducted as described under the examples 1-5.

Precursor (sequence): Glu-(urea)-Lys-2Nal*- Inp-βAla-ser-ser-ser-MA (*=bound to the s-amino group of the neighboring lysine; 2Nal=L-2-Naphtylalanine; Inp=isonipecotic acid; βAla=Beta-alanine; MA=2-Mercaptoacetic acid)

Chemical formula: C₄₅H₆₃N₉O₁₇S (Mw: 1034.10 g/mol)

MS (HPLC-ESI-MS): m/z ([M+H⁺]⁺)=1034.39 (found); 1034.41 (calc.); tᵣ=11.7 min (gradient A, see example 9)

Purity (HPLC): >95%, tᵣ=2.147 min (gradient B, see example 9)

### [¹⁸⁸Re]Re-GCK05

RCY/RCP: 81%/>90%

HPLC (gradient B, see Example 9): tᵣ=2.210 min

### [^{99m}Tc]Tc-GCK05

RCY/RCP: >95%

HPLC (gradient B, see Example 9): tᵣ=2.203 min

### Example 13: GCK06

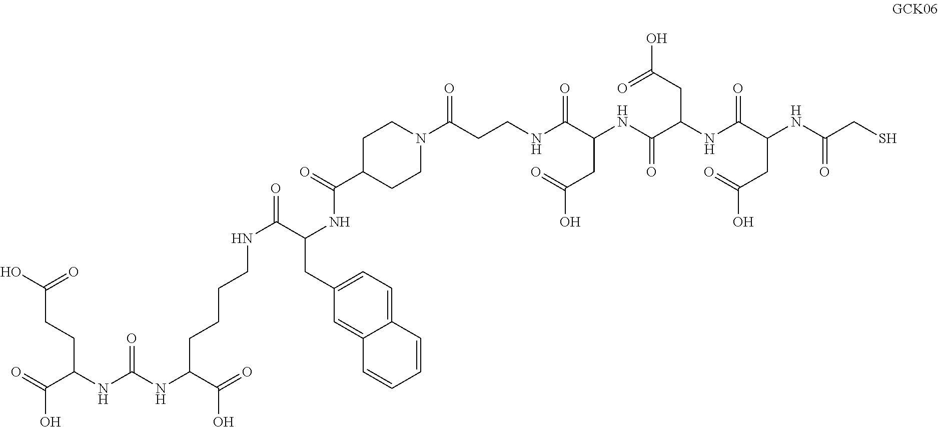 [SMILES: O=C(O)CCC(NC(=O)NC(CCCCNC(=O)C(Cc1ccc2ccccc2c1)NC(=O)C1CCN(C(=O)CCNC(=O)C(CC(=O)O)NC(=O)C(CC(=O)O)NC(=O)C(CC(=O)O)NC(=O)CS)CC1)C(=O)O)C(=O)O]

All syntheses were conducted as described under the examples 1-5.

Precursor (sequence): Glu-(urea)-Lys-2Nal*-Inp-βAla-asp-asp-asp-MA (*=bound to the ε-amino group of the neighboring lysine; 2Nal=L-2-Naphtylalanine; Inp=isonipecotic acid; βAla=Beta-alanine; MA=2-Mercaptoacetic acid)

Chemical formula: C₄₈H₆₃N₉O₂₀S (Mw: 1118.13 g/mol)

MS (HPLC-ESI-MS): m/z ([M+H⁺]⁺)=1118.37 (found); 1118.40 (calc.); tᵣ=11.9 min (gradient A, see example 9)

Purity (HPLC): >95%, tᵣ=2.173 min (gradient B, see example 9)

### [¹⁸⁸Re]Re-GCK06

RCY/RCP: 66%/>90%

HPLC (gradient B, see Example 9): tᵣ=2.278 min

### [^{99m}Tc]Tc-GCK06

RCY/RCP: >98%

HPLC (gradient B, see Example 9): tᵣ=2.237 min

### Example 14: GCK07

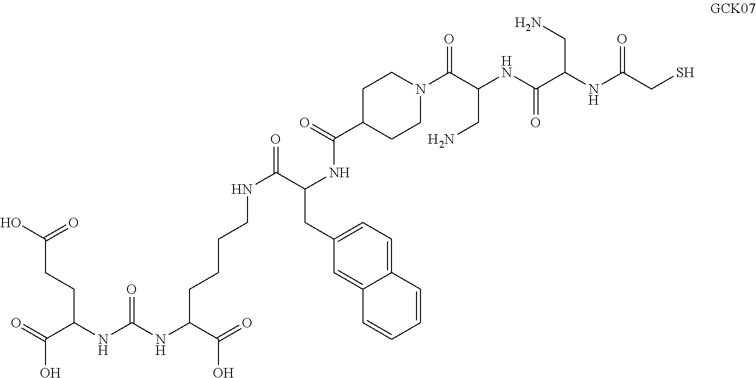 [SMILES: NCC(NC(=O)CS)C(=O)NC(CN)C(=O)N1CCC(C(=O)NC(Cc2ccc3ccccc3c2)C(=O)NCCCCC(NC(=O)NC(CCC(=O)O)C(=O)O)C(=O)O)CC1]

All syntheses were conducted as described under the examples 1-5.

Precursor (sequence): Glu-(urea)-Lys-2Nal*-Inp-dap-dap-MA (*=bound to the ε-amino group of the neighboring lysine; 2Nal=L-2-Naphtylalanine; Inp=isonipecotic acid; dap=2,3-diaminopropionic acid; MA=2-Mercaptoacetic acid)

Chemical formula: C₃₉H₅₅N₉O₁₂S (Mw: 873.97 g/mol)

Purity (HPLC): >95%, tᵣ=2.010 min (gradient B, see example 9)

### [^{99m}Tc]Tc-GCK07

RCY/RCP: >98%

HPLC (gradient B, see Example 9): tᵣ=2.260 min

### Example 15: GCK09

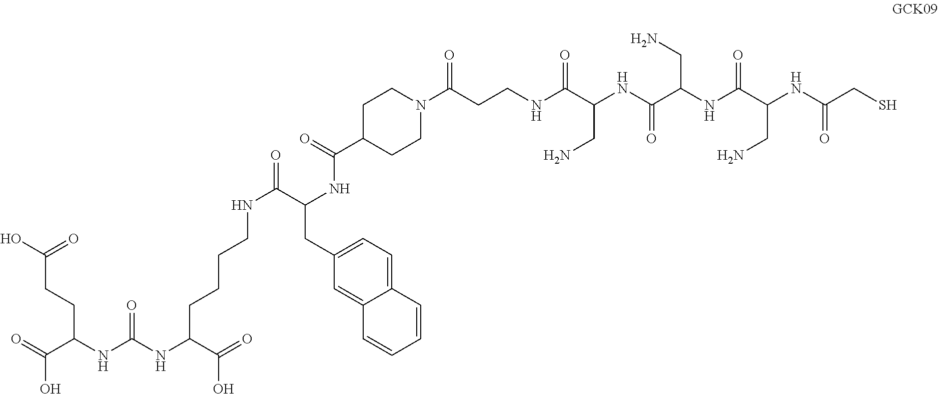 [SMILES: NCC(NC(=O)CS)C(=O)NC(CN)C(=O)NC(CN)C(=O)NCCC(=O)N1CCC(C(=O)NC(Cc2ccc3ccccc3c2)C(=O)NCCCCC(NC(=O)NC(CCC(=O)O)C(=O)O)C(=O)O)CC1]

All syntheses were conducted as described under the examples 1-5.

Precursor (sequence): Glu-(urea)-Lys-2Nal*-Inp-βAla-dap-dap-MA (*=bound to the ε-amino group of the neighboring lysine; 2Nal=L-2-Naphtylalanine; Inp=isonipecotic acid; βAla=Beta-alanine; dap=2,3-diaminopropionic acid; MA=2-Mercaptoacetic acid)

Chemical formula: C₄₅H₆₆N₁₂O₁₄S (Mw: 1031.14 g/mol)

MS (HPLC-ESI-MS): m/z=1031.471 (found); 1031.462 (calc.); tᵣ=10.99 min (gradient A, see example 9)

Purity (HPLC): >90%, tᵣ=2.051 min (gradient B, see example 9)

### [¹⁸⁸Re]Re-GCK09

RCY/RCP: 81%/>80%

HPLC (gradient B, see Example 9): tᵣ=2.063 min

### [^{99m}Tc]Tc-GCK09

RCY/RCP: >90%

HPLC (gradient B, see Example 9): tᵣ=2.085 min

### Example 16: GCK11

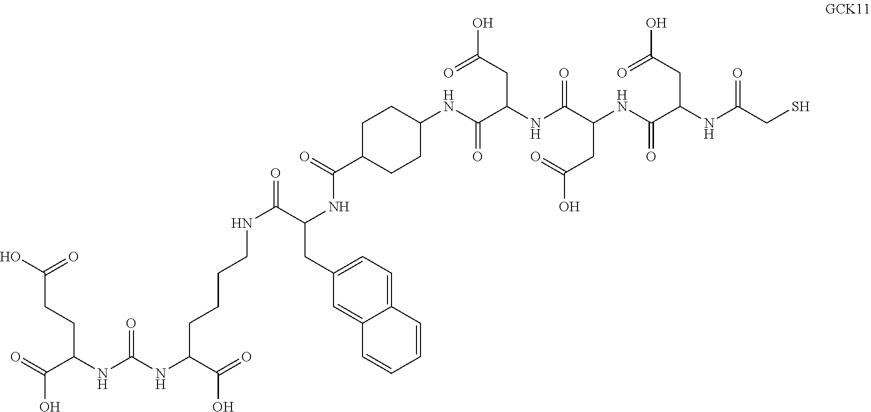 [SMILES: O=C(O)CCC(NC(=O)NC(CCCCNC(=O)C(Cc1ccc2ccccc2c1)NC(=O)C1CCC(NC(=O)C(CC(=O)O)NC(=O)C(CC(=O)O)NC(=O)C(CC(=O)O)NC(=O)CS)CC1)C(=O)O)C(=O)O]

All syntheses were conducted as described under the examples 1-5.

Precursor (sequence): Glu-(urea)-Lys-2Nal*-tACHC-asp-asp-asp-MA (*=bound to the s-amino group of the neighboring lysine; 2Nal=L-2-Naphtylalanine; tACHC=trans-4-aminocyclohexane carboxylic acid; MA=2-Mercaptoacetic acid)

Chemical formula: C₄₆H₆₀N₈O₁₉S (Mw: 1061.08 g/mol)

MS (HPLC-ESI-MS): m/z=1061.345 (found); 1061.377 (calc.); tᵣ=12.04 min (gradient A, see example 9)

Purity (HPLC): >95%, tᵣ=2.219 min (gradient B, see example 9)

### [^{99m}Tc]Tc-GCK11

RCY/RCP: >95%

HPLC (gradient B, see Example 9): tᵣ=2.361 min

### Example 17: GCK13

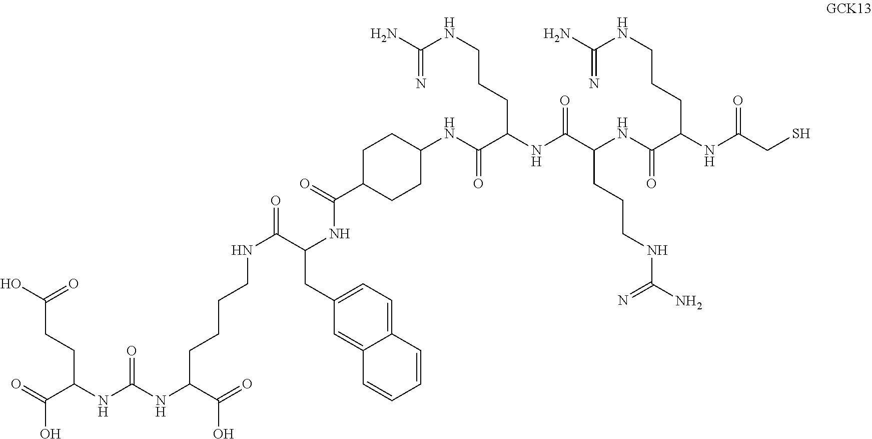 [SMILES: N=C(N)NCCCC(NC(=O)CS)C(=O)NC(CCCNC(=N)N)C(=O)NC(CCCNC(=N)N)C(=O)NC1CCC(C(=O)NC(Cc2ccc3ccccc3c2)C(=O)NCCCCC(NC(=O)NC(CCC(=O)O)C(=O)O)C(=O)O)CC1]

All syntheses were conducted as described under the examples 1-5.

Precursor (sequence): Glu-(urea)-Lys-2Nal*-tACHC-arg-arg-arg-MA (*=bound to the s-amino group of the neighboring lysine; 2Nal=L-2-Naphtylalanine; tACHC=trans-4-aminocyclohexane carboxylic acid; MA=2-Mercaptoacetic acid)

Chemical formula: C₅₂H₈₁N₁₇O₁₃S (Mw: 1184.37 g/mol)

MS (HPLC-ESI-MS): m/z=1184.574 (found); 1184.600 (calc.); tᵣ=10.98 min (gradient A, see example 9)

Purity (HPLC): >90%, tᵣ=2.294 min (gradient B, see example 9)

### [¹⁸⁸Re]Re-GCK13

RCY/RCP: 79%/>90%

HPLC (gradient B, see Example 9): tᵣ=2.234 min

### [^{99m}Tc]Tc-GCK13

RCY/RCP: >90%

HPLC (gradient B, see Example 9): tᵣ=2.382 min

1. A compound of formula (I) or a stereoisomer or tautomer, thereof, 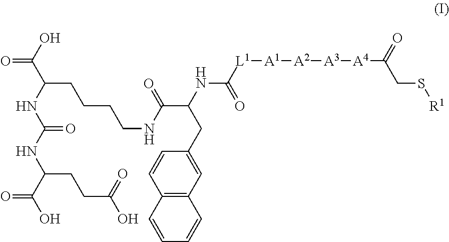
wherein,
R¹ is selected from the group consisting of hydrogen, —C(O)R¹¹, —C(O)₂R¹², —C(O)NR¹²R¹³, —SR¹⁰, C₁₋₆alkyl, arylC₁₋₆alkyl, heteroarylC₁₋₆alkyl, heterocyclylC₁₋₆alkyl, aryl, heteroaryl, heterocyclyl; wherein said C₁₋₆alkyl, arylC₁₋₆alkyl, heteroarylC₁₋₆alkyl, heterocyclylC₁₋₆alkyl, aryl, heteroaryl, or heterocyclyl can be unsubstituted or substituted with one or more Z¹;
L¹ is 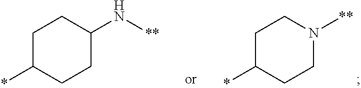
wherein * represents where L¹ is bound to the carbonyl group; and ** represents where L¹ is bound to A¹;
A¹ is 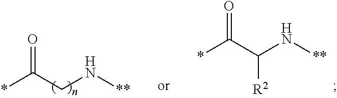
wherein * represents where A¹ is bound L¹; and ** represents where A¹ is bound to A²; and wherein,
n is an integer selected from 1, 2, 3, 4 or 5;
R² is selected from the group consisting of hydroxyC₁₋₆alkyl, C₁₋₆alkylNHR³, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂;
R³ is H or —C(O)(CH₂)SH;
A² is selected from: 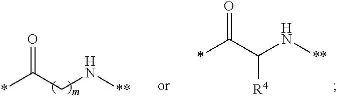
wherein * represents where A² is bound A¹; and ** represents where A² is bound to A³; or wherein A³ is absent; and wherein,
m is an integer selected from 1, 2, 3, 4 or 5;
R⁴ is selected from the group consisting of hydroxyC₁₋₆alkyl, C₁₋₆alkylNHR⁵, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂;
R⁵ is H or —C(O)(CH₂)SH;
A³ is selected from: 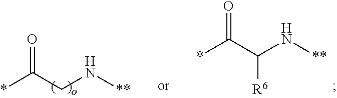
wherein * represents where A³ is bound A²; and ** represents where A³ is bound to A⁴; or wherein A³ is absent; and wherein,
o is an integer selected from 1, 2, 3, 4 or 5;
R⁶ is selected from the group consisting of hydroxyC₁₋₆alkyl, C₁₋₆alkylNHR⁷, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₆alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂;
R⁷ is H or —C(O)(CH₂)SH;
A⁴ is selected from: 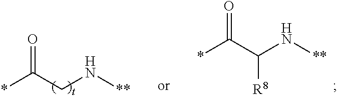
wherein * represents where A⁴ is bound A³; and ** represents where A⁴ is bound to the carbonyl group; or wherein A⁴ is absent; and wherein,
t is an integer selected from 1, 2, 3, 4 or 5;
R⁸ is selected from the group consisting of hydroxyC₁₋₆alkyl, C₁₋₆alkylNHR⁹, C₁₋₆alkylC(O)OH, C₁₋₆alkylNHC(NH)NH₂, hydrogen, C₁₋₆alkyl, C₂₋₆alkenyl, aminoC₁₋₆alkyl, mercaptoC₁₋₆alkyl, C₁₋₆alkylthioC₁₋₆alkylene, arylC₁₋₄alkyl, —CH(OH)CH₃, —C(O)OH, C₁₋₆alkyl(CO₂H)₂, —SO₃H, C₁₋₆alkylheteroaryl, C₁₋₆alkylSeH, C₁₋₆alkylS(O)CH₃, C₁₋₆alkylS(CH₃)₂⁺, C₁₋₆alkylNHC(O)heterocycle and C₁₋₆alkylC(O)NH₂;
R⁹ is H or —C(O)(CH₂)SH;
R¹⁰ is selected from the group consisting of C₁₋₆alkyl, heterocycle, aryl, and heteroaryl;
or R¹⁰ is a group of formula (i); 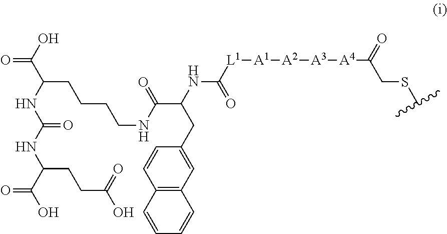
wherein the wavy line ( [symbol] ) indicates the point of attachment to the S atom and L¹, A¹, A², A³, and A⁴ are as defined for structure (I);
each R¹¹ is independently selected from the group consisting of C₁₋₆alkyl, haloC₁₋₆alkyl, aryl, haloC₁₋₄alkyl, arylC₁₋₆alkyl, heterocyclyl, heteroaryl;
each R¹² is independently selected from the group consisting of hydrogen, C₁₋₆alkyl, aryl, haloC₁₋₆alkyl, CH₂CCl₃, CH₂OCH₃, arylC₁₋₆alkyl, heterocyclyl, heteroaryl;
each R¹³ is independently selected from the group consisting of hydrogen, C₁₋₆alkyl, aryl, haloC₁₋₆alkyl, arylC₁₋₆alkyl, heterocyclyl, heteroaryl;
each Z¹ is independently selected from the group consisting of —OR¹¹, —C(O)R¹¹, nitro, hydroxyl, C₁₋₆alkyl, aryl, heteroaryl, —SR¹¹, —NR¹²C(O)R¹³, —C(O)₂R¹², cyano, —S(O)₂R¹⁰, halo, haloC₁₋₆alkyl, haloC₁₋₆alkyloxy, heterocyclyl, amino, —NR¹¹R¹², —C(O)NR¹²R¹³, —S(O)R¹⁰, —S(O)₂N R¹²R¹³; wherein said C₁₋₆alkyl, or aryl, can be unsubstituted or substituted with one or more C₁₋₄alkyl, methoxy, nitro, —C(O)aryl, halo, trifluoromethyl, trifluoromethoxy;
or a solvate, hydrate, salt or prodrug thereof.

2. The compound according to claim 1, having structural formula (IA), 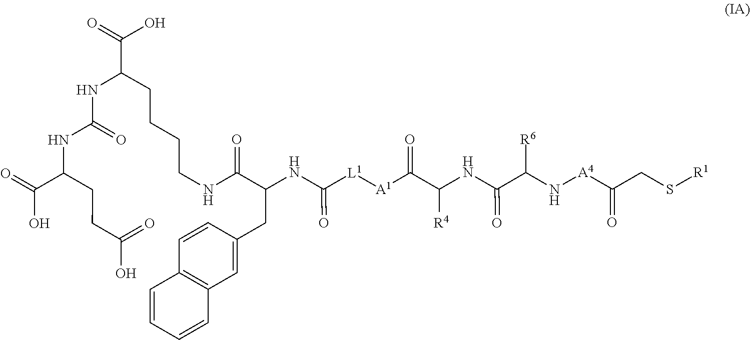
wherein L¹, A¹, A⁴, R¹, R⁴, and R⁶ have the same meaning as that defined in claim 1.

3. The compound according to claim 2, having structural formula (IB), 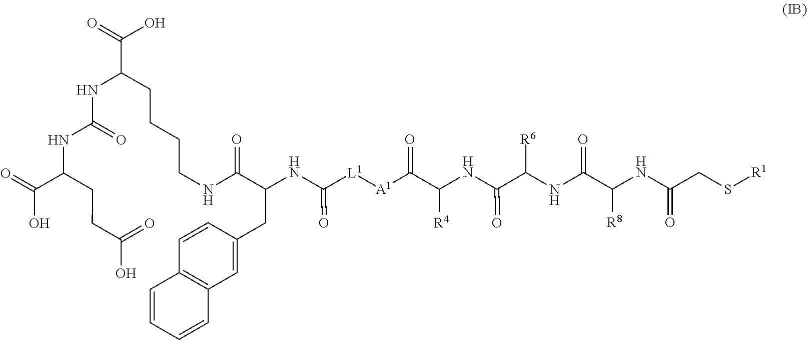
wherein L¹, A¹, R¹, R⁴, R⁶, and R⁸ have the same meaning as that defined in claim 1.

4. The compound according to claim 1, wherein,
R² is selected from the group consisting of —CH₂OH, —CH₂NHR³, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R⁴ is selected from the group consisting of —CH₂OH, —CH₂NHR⁵, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R⁶ is selected from the group consisting of —CH₂OH, —CH₂NHR⁷, —CH₂C(O)OH, —(CH₂)₃NHC(NH)NH₂, hydrogen, C₁₋₆alkyl, —(CH₂)₂OH, —(CH₂)₄NH₂, —CH₂SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂;
R⁸ is selected from the group consisting of —CH₂OH, —CH₂NHR⁹, —CH₂C(O)OH, —(CH₂)₃NHC(NH)NH₂, hydrogen, C₁₋₆alkyl, —(CH₂)₂OH, —(CH₂)₄NH₂, —CH2SH, —(CH₂)₂SCH₃, arylC₁₋₆alkyl, —CH(OH)CH₃, —C(O)OH, —SO₃H, C₁₋₆alkylheteroaryl, —CH₂C(O)NH₂, and —(CH₂)₂C(O)NH₂; preferably wherein, R¹ is hydrogen, acetyl or —SR¹⁰, wherein R¹⁰ is a group of formula (i).

5. The compound claim 1, selected from the group consisting of:

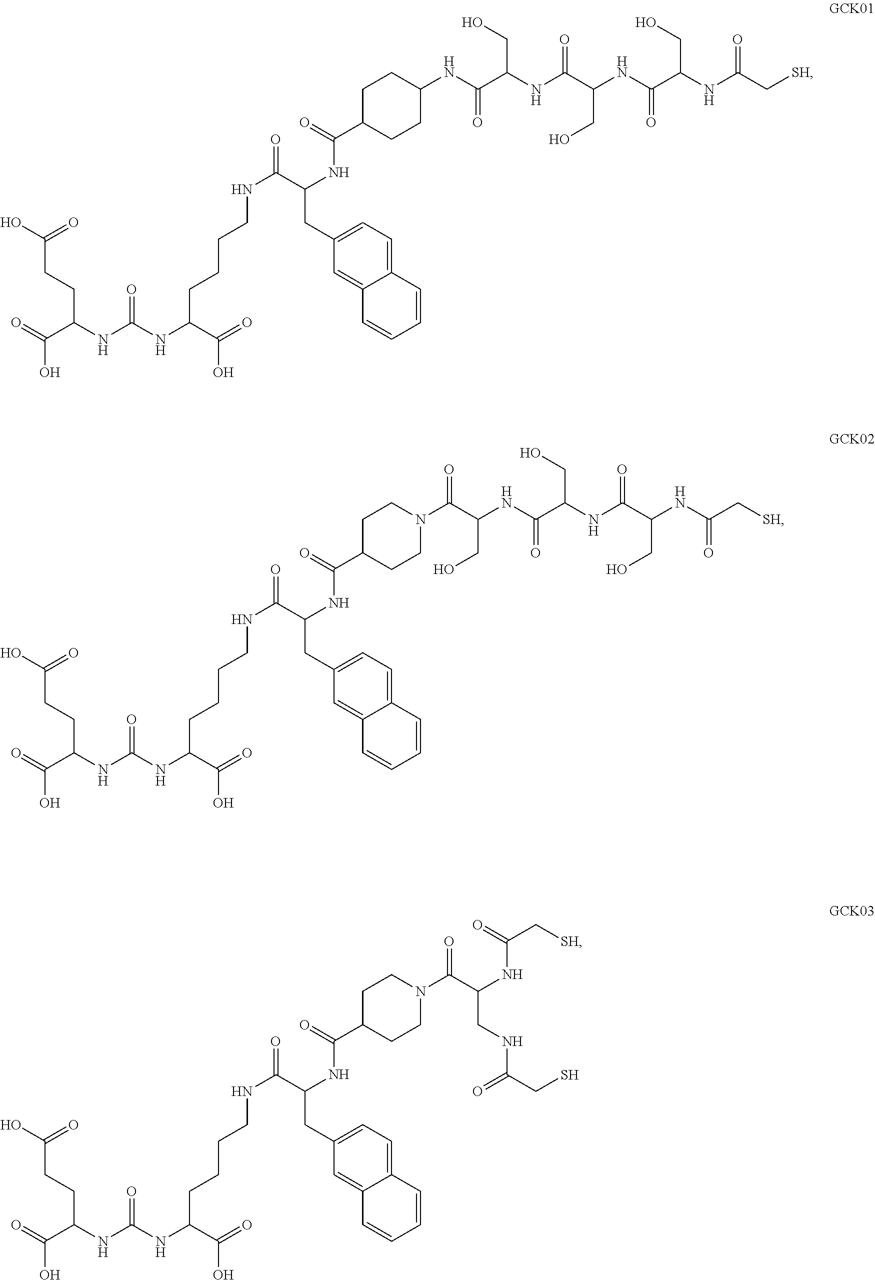 [SMILES: O=C(O)CCC(NC(=O)NC(CCCCNC(=O)C(Cc1ccc2ccccc2c1)NC(=O)C1CCC(NC(=O)C(CO)NC(=O)C(CO)NC(=O)C(CO)NC(=O)CS)CC1)C(=O)O)C(=O)O ; O=C(O)CCC(NC(=O)NC(CCCCNC(=O)C(Cc1ccc2ccccc2c1)NC(=O)C1CCN(C(=O)C(CO)NC(=O)C(CO)NC(=O)C(CO)NC(=O)CS)CC1)C(=O)O)C(=O)O ; O=C(O)CCC(NC(=O)NC(CCCCNC(=O)C(Cc1ccc2ccccc2c1)NC(=O)C1CCN(C(=O)C(CNC(=O)CS)NC(=O)CS)CC1)C(=O)O)C(=O)O]

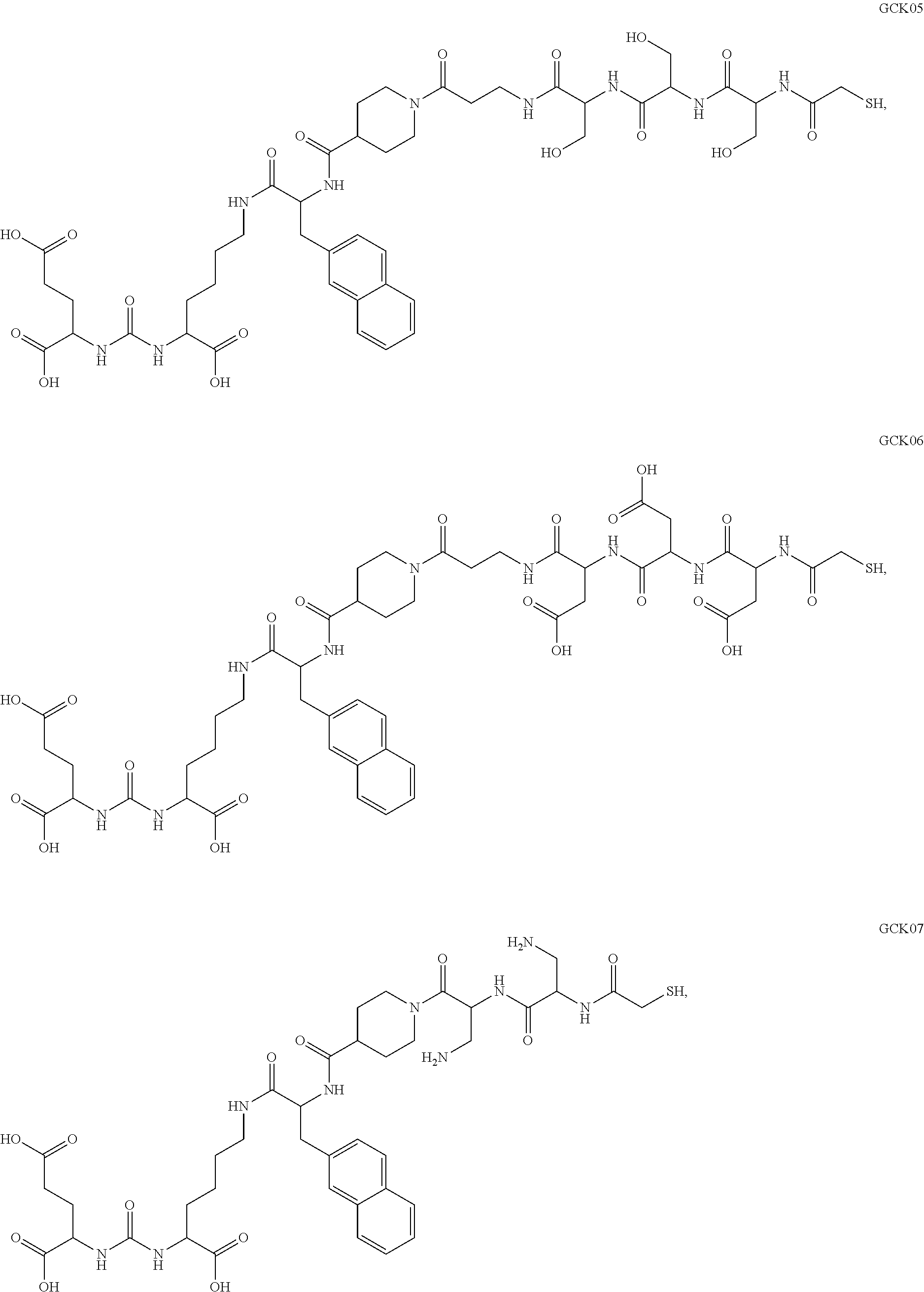 [SMILES: O=C(O)CCC(NC(=O)NC(CCCCNC(=O)C(Cc1ccc2ccccc2c1)NC(=O)C1CCN(C(=O)CCNC(=O)C(CO)NC(=O)C(CO)NC(=O)C(CO)NC(=O)CS)CC1)C(=O)O)C(=O)O ; O=C(O)CCC(NC(=O)NC(CCCCNC(=O)C(Cc1ccc2ccccc2c1)NC(=O)C1CCN(C(=O)CCNC(=O)C(CC(=O)O)NC(=O)C(CC(=O)O)NC(=O)C(CC(=O)O)NC(=O)CS)CC1)C(=O)O)C(=O)O ; NCC(NC(=O)CS)C(=O)NC(CN)C(=O)N1CCC(C(=O)NC(Cc2ccc3ccccc3c2)C(=O)NCCCCC(NC(=O)NC(CCC(=O)O)C(=O)O)C(=O)O)CC1]

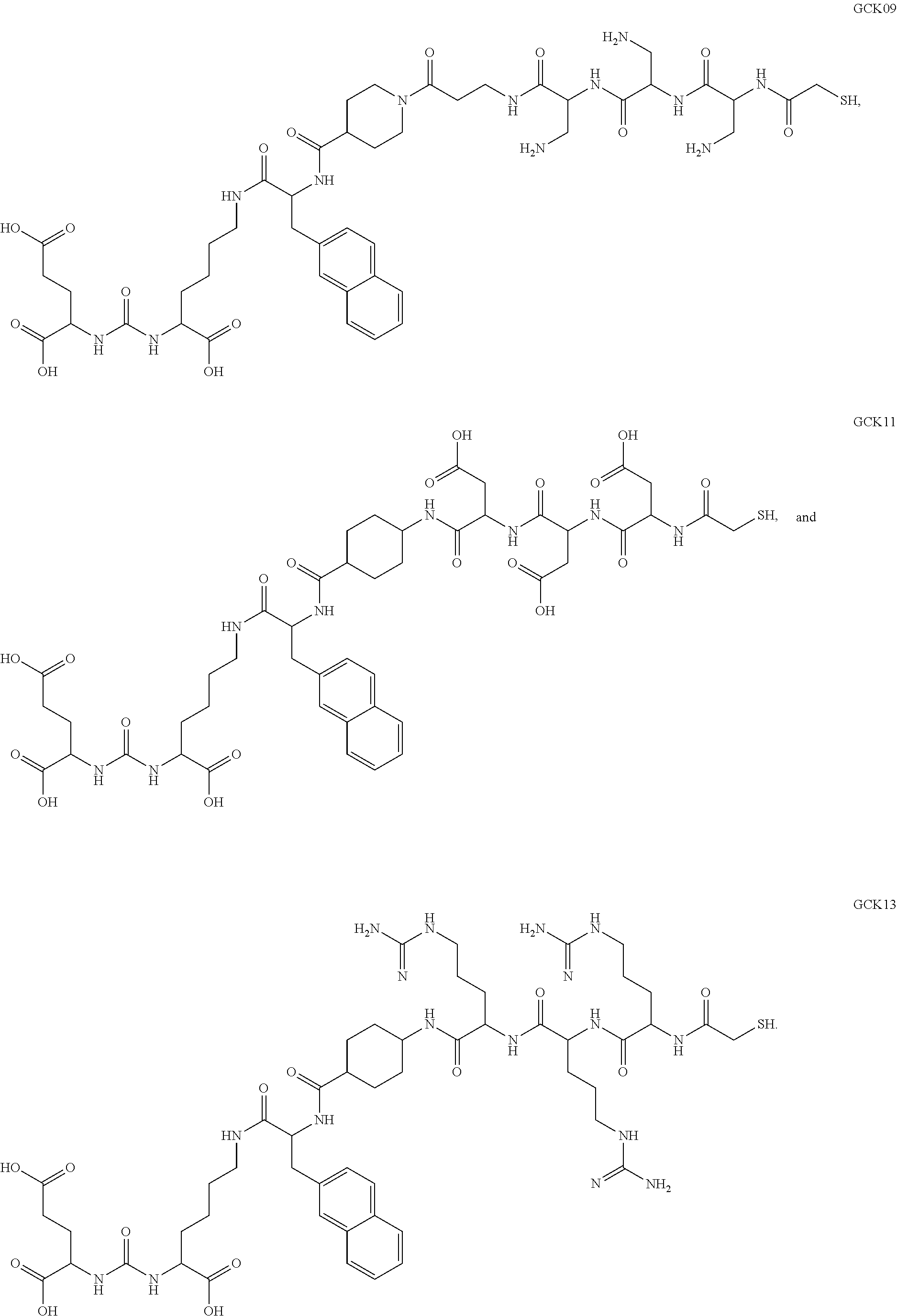 [SMILES: NCC(NC(=O)CS)C(=O)NC(CN)C(=O)NC(CN)C(=O)NCCC(=O)N1CCC(C(=O)NC(Cc2ccc3ccccc3c2)C(=O)NCCCCC(NC(=O)NC(CCC(=O)O)C(=O)O)C(=O)O)CC1 ; O=C(O)CCC(NC(=O)NC(CCCCNC(=O)C(Cc1ccc2ccccc2c1)NC(=O)C1CCC(NC(=O)C(CC(=O)O)NC(=O)C(CC(=O)O)NC(=O)C(CC(=O)O)NC(=O)CS)CC1)C(=O)O)C(=O)O ; N=C(N)NCCCC(NC(=O)CS)C(=O)NC(CCCNC(=N)N)C(=O)NC(CCCNC(=N)N)C(=O)NC1CCC(C(=O)NC(Cc2ccc3ccccc3c2)C(=O)NCCCCC(NC(=O)NC(CCC(=O)O)C(=O)O)C(=O)O)CC1]

6. A solvate, hydrate, salt or prodrug of the compound of claim 1.

7. A metal complex comprising a compound of formula (I) according to claim 1, and an element of Group VII of the Periodic Table.

8. A metal complex according to claim 7 wherein the element is a radionuclide.

9. A metal complex according to claim 7 wherein the element is ^{99m}Tc or ¹⁸⁸Re or ¹⁸⁶Re.

10. A pharmaceutical composition comprising one or more pharmaceutically acceptable excipients and a metal complex according to claim 7.

11-15. (canceled)

16. A method of treating or preventing cancer in a subject in need thereof comprising administering to said subject a therapeutically effective amount of the pharmaceutical composition according to claim 10.

17. The method according to claim 16, wherein the radionuclide is ¹⁸⁸Re or ¹⁸⁶Re.

18. A method of in-vivo imaging or detection of tumor or cancer cells or of in-vivo diagnosis of cancer in a subject, comprising administering a suitable amount of the pharmaceutical composition according to claim 10 to said subject and visualizing said metal complex using an in-vivo radio-imaging method.

19. The method according to claim 18, wherein the imaging method is positron emission tomography (PET), PET computed tomography (PET-CT) or single-photon emission tomography (SPECT).

20. The method according to claim 18, wherein the radionuclide used for imaging is ^{99m}Tc.

21. The method according to claim 16, wherein said cancer is a PSMA-expressing cancer or tumor, a conventional renal cell cancer, transitional cell of the bladder cancer, non-small-cell lung cancer, testicular-embryonal cancer, neuroendocrine cancer, colon cancer, prostate cancer, or breast cancer.

22. A radiolabeling kit comprising:
the compound according to claim 1,
a suitable buffering system, preferably selected from the group consisting of: phosphate buffers, acetate buffers, formate buffers, and HEPES buffers, more preferably phosphate buffers, even more preferably a sodium-phosphate buffer; and
a suitable reducing agent, enabling the reduction of the pertechnetate/perrhenate to Tc(V)O/Re(V)O, such as but not limited to: ascorbic acid, sodium borohydride, sodium dithionite, phosphines such as TCEP, and stannous chloride (Tin(II)chloride), preferably stannous chloride most preferably stannous chloride (tin(II)chloride).

23. The radiolabelling kit according to claim 22, further comprising any one or more of:
a suitable anti-oxidant agent such as but not limited to: sodium ascorbate/ascorbic acid mixtures, sodium borohydride, sodium dithionite, and stannous chloride,
a suitable auxiliary agent or ligand enabling the protection against reoxidation of Tc(V)O/Re(V)O as competing reaction to coordination, such as but not limited to: tartrate, citrate or glucoheptonate,
a sequestering agent competing with the chelator for radiometal impurities
a stabilizer enabling the storage of the kit, and/or
an excipient such as lyophilization agent, matrix reagent or solubilizer.

24. A method of radiolabelling a compound according to claim 1, comprising the steps of:
providing a compound or labelling precursor according to claim 1,
providing a suitable buffering system
providing a radionuclide, preferably selected from ^{99m}Tc or ¹⁸⁸Re and ¹⁸⁶Re
providing a suitable reducing agent
mixing all components at a suitable pH and allowing the complexation of the radionuclide and labelling precursor to occur, thereby obtaining a radiolabelled compound.

25. The method of claim 24, wherein said buffering system is selected from the group consisting of: phosphate buffers, acetate buffers, formate buffers, and HEPES buffers, more preferably phosphate buffers, even more preferably a sodium-phosphate buffer.

26. The method of claim 24, wherein when the radionuclide used is ^{99m}Tc, the precursor and buffer are mixed and a suitable amount of pertechnetate is eluted in saline from a molybdenum-99 (⁹⁹Mo/⁹⁹Tc) generator into said mixture.

27. The method of claim 24, wherein when the radionuclide used is ¹⁸⁸Re, the precursor and buffer are mixed and a suitable amount of Rhenium is eluted in saline from a tungsten-188 (¹⁸⁸W/¹⁸⁸Re) generator into said mixture.

28. The method of claim 24, wherein when the radionuclide used is ¹⁸⁶Re, the precursor and buffer are mixed and a suitable amount of Rhenium-186 is produced from a cyclotron or reactor and added into said mixture.

29-31. (canceled)
